(19) United States
(12) Patent Application Publication
(10) Pub. No.: US 2010/0256146 A1
(43) Pub. Date: Oct. 7, 2010

(54) TRISUBSTITUTED PIPERIDINES

(75) Inventors: Peter Herold, Munchenstein (CH); Vincenzo Tschinke, Binningen (CH); Dirk Behnke, Grenzach-Whylen (DE); Stjepan Jelakovic, Freiburg (DE); Nathalie Jotterand, Basel (CH); Robert Mah, Muttenz (CH)

Correspondence Address: WENDEROTH, LIND & PONACK, L.L.P., 1030 15th Street, N.W.,, Suite 400 East, Washington, DC, 20005-1503, US

(73) Assignee: Novartis AG

(21) Appl. No.: 12/734,197

(22) PCT Filed: Oct. 17, 2008
(86) PCT No.: PCT/EP2008/064010

§ 371 Date: Apr. 16, 2010

(30) Foreign Application Priority Data
Oct. 18, 2007 (EP) 07118823.9

(51) Int. Cl. A61K 31/445; C07D 413/02; A61P 9/00; A61P 13/12

(52) U.S. Cl. 514/235.5; 544/129

(57) ABSTRACT

The application relates to trisubstituted piperidines of the general formula (I) and their salts, preferably their pharmaceutically acceptable salts, in which R¹, R², R³ and X have the meanings explained in the description, a process for their preparation and the use of these compounds as medicines, especially as renin inhibitors.

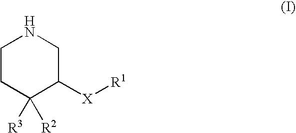

### FIELD OF THE INVENTION

The present invention relates to novel trisubstituted piperidines, processes for their preparation and the use of the compounds as medicines, especially as renin inhibitors.

### BACKGROUND OF THE INVENTION

Piperidine derivatives for use as medicines are disclosed for example in WO 97/09311. However, especially with regard to renin inhibition, there continues to be a need for highly potent active ingredients. In this context, the improvement of a compound's pharmacokinetic properties, resulting in better oral bioavailability, and/or it's overall safety profile are at the forefront. Properties directed towards better bioavailability are, for example, increased absorption, metabolic stability or solubility, or optimized lipophilicity. Properties directed towards a better safety profile are, for example, increased selectivity against drug metabolizing enzymes such as the cytochrome P450 enzymes. Moreover, especially with regard to renin inhibitors, an additional consideration are efforts to simplify the manufacturing process through a reduction in the number of chemical steps/processes, thus resulting in a reduction in the cost of goods.

### DETAILED DESCRIPTION OF THE INVENTION

The invention therefore relates firstly to trisubstituted piperidines of the general formula

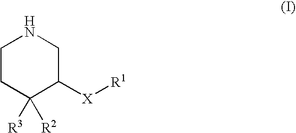

in which

R¹ is aryl or heterocyclyl, each of which is substituted by 1-4 radicals independently selected from the group consisting of

acyl-C₁₋₈-alkoxy-C₁₋₈-alkoxy,

acyl-C₁₋₈-alkoxy-C₁₋₈-alkyl,

(N-acyl)-C₁₋₈-alkoxy-C₁₋₈-alkylamino,

C₁₋₈-alkanoyl,

C₁₋₈-alkoxy,

C₁₋₈-alkoxy-C₁₋₈-alkanoyl,

C₁₋₈-alkoxy-C₁₋₈-alkoxy,

C₁₋₈-alkoxy-C₁₋₈-alkoxy-C₁₋₈-alkyl,

C₁₋₈-alkoxy-C₁₋₈-alkyl,

(N—C₁₋₈-alkoxy)-C₁₋₈-alkylaminocarbonyl-C₁₋₈-alkoxy,

(N—C₁₋₈-alkoxy)-C₁₋₈-alkylaminocarbonyl-C₁₋₈-alkyl,

C₁₋₈-alkoxy-C₁₋₈-alkylcarbamoyl,

C₁₋₈-alkoxy-C₁₋₈-alkylcarbonyl,

C₁₋₈-alkoxy-C₁₋₈-alkylcarbonylamino,

C₁₋₈-alkoxy-C₁₋₈-alkylheterocyclyl,

C₁₋₈-alkoxycarbonyl,

C₁₋₈-alkoxycarbonyl-C₁₋₈-alkoxy,

C₁₋₈-alkoxycarbonyl-C₁₋₈-alkyl,

C₁₋₈-alkoxycarbonylamino-C₁₋₈-alkoxy,

C₁₋₈-alkoxycarbonylamino-C₁₋₈-alkyl,

C₁₋₈-alkyl,

(N—C₁₋₈-alkyl)-C₁₋₈-alkoxy-C₁₋₈-alkylcarbamoyl,

(N—C₁₋₈-alkyl)-C₁₋₈-alkoxy-C₁₋₈-alkylcarbonylamino,

(N—C₁₋₈-alkyl)-C₁₋₈-alkoxycarbonylamino,

(N—C₁₋₈-alkyl)-C₁₋₈-alkylcarbonylamino-C₁₋₈-alkoxy,

(N—C₁₋₈-alkyl)-C₁₋₈-alkylcarbonylamino-C₁₋₈-alkyl,

(N—C₁₋₈-alkyl)-C₁₋₈-alkylsulfonylamino-C₁₋₈-alkoxy,

(N—C₁₋₈-alkyl)-C₁₋₈-alkylsulfonylamino-C₁₋₈-alkyl,

C₁₋₈-alkylamidinyl,

C₁₋₈-alkylamino-C₁₋₈-alkoxy,

di-C₁₋₈-alkylamino-C₁₋₈-alkoxy,

C₁₋₈-alkylamino-C₁₋₈-alkyl,

di-C₁₋₈-alkylamino-C₁₋₈-alkyl,

C₁₋₈-alkylaminocarbonyl-C₁₋₈-alkoxy,

di-C₁₋₈-alkylaminocarbonyl-C₁₋₈-alkoxy,

C₁₋₈-alkylaminocarbonyl-C₁₋₈-alkoxy-C₁₋₈-alkyl,

C₁₋₈-alkylaminocarbonyl-C₁₋₈-alkyl,

di-C₁₋₈-alkylaminocarbonyl-C₁₋₈-alkyl,

C₁₋₈-alkylaminocarbonylamino-C₁₋₈-alkoxy,

C₁₋₈-alkylaminocarbonylamino-C₁₋₈-alkyl,

C₀₋₈-alkylcarbonylamino,

C₀₋₈-alkylcarbonylamino-C₁₋₈-alkoxy,

C₀₋₈-alkylcarbonylamino-C₁₋₈-alkyl,

C₁₋₈-alkylcarbonyloxy-C₁₋₈-alkoxy,

C₁₋₈-alkylcarbonyloxy-C₁₋₈-alkyl,

C₁₋₈-alkylsulfonyl,

C₁₋₈-alkylsulfonyl-C₁₋₈-alkoxy,

C₁₋₈-alkylsulfonyl-C₁₋₈-alkyl,

C₁₋₈-alkylsulfonylamino-C₁₋₈-alkoxy,

C₁₋₈-alkylsulfonylamino-C₁₋₈-alkyl,

optionally N-mono- or N,N-di-C₁₋₈-alkylated amino,

aryl-C₀₋₈-alkoxy,

aryl-C₀₋₈-alkyl,

optionally N-mono- or N,N-di-C₁₋₈-alkylated carbamoyl-C₀₋₈-alkoxy,

optionally N-mono- or N,N-di-C₁₋₈-alkylated carbamoyl-C₀₋₈-alkyl,

carboxy-C₁₋₈-alkoxy,

carboxy-C₁₋₈-alkoxy-C₁₋₈-alkyl,

carboxy-C₁₋₈-alkyl,

cyano,

cyano-C₁₋₈-alkoxy,

cyano-C₁₋₈-alkyl,

C₃₋₁₂-cycloalkyl-C₁₋₈-alkoxy,

C₃₋₁₂-cycloalkyl-C₁₋₈-alkyl,

C₃₋₁₂-cycloalkylcarbonylamino-C₁₋₈-alkoxy,

C₃₋₁₂-cycloalkylcarbonylamino-C₁₋₈-alkyl,

O,N-dimethylhydroxylamino-C₁₋₈-alkyl,

halogen,

halogen substituted C₁₋₈-alkoxy,

halogen substituted C₁₋₈-alkyl,

heterocyclyl-C₀₋₈-alkoxy,

heterocyclyl-C₀₋₈-alkyl,

heterocyclylcarbonyl,

hydroxy-C₁₋₈-alkoxy-C₁₋₈-alkoxy,

hydroxy-C₁₋₈-alkoxy-C₁₋₈-alkyl,

hydroxy-C₁₋₈-alkyl,

oxide and oxo;

where, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, where one CH₂ group of the alkylene chain may be replaced by oxygen;

R² is phenyl or pyridyl, where the nitrogen atom of the pyridyl is located in the ortho- or meta-position relative to the bond from the pyridyl ring to the remainder of the molecule and where the phenyl or pyridyl is substituted by 1-3 radicals, preferably one of which is located in the para-position relative to the bond from the phenyl or pyridyl ring to the remainder of the molecule, independently selected from the group consisting of

C₁₋₈-alkanoyloxy-C₁₋₈-alkyl,

C₂₋₈-alkenyl,

C₂₋₈-alkenyloxy,

C₂₋₈-alkenyloxy-C₁₋₈-alkyl,

C₁₋₈-alkoxy,

C₁₋₈-alkoxy-C₁₋₈-alkoxy,

C₁₋₈-alkoxy-C₁₋₈-alkoxy-C₁₋₈-alkoxy,

C₁₋₈-alkoxy-C₁₋₈-alkoxy-C₁₋₈-alkoxy-C₁₋₈-alkyl,

C₁₋₈-alkoxy-C₁₋₈-alkoxy-C₁₋₈-alkyl,

C₁₋₈-alkoxy-C₁₋₈-alkyl,

C₁₋₈-alkoxy-C₁₋₈-alkylamino-C₁₋₈-alkyl,

C₁₋₈-alkoxy-C₀₋₈-alkyl-C₃₋₈-cycloalkyl-C₀₋₈-alkoxy-C₁₋₈-alkyl,

C₁₋₈-alkoxy-C₁₋₈-alkylsulfanyl,

C₁₋₈-alkoxy-C₁₋₈-alkylsulfanyl-C₁₋₈-alkyl,

C₁₋₈-alkoxycarbonyl,

C₁₋₈-alkoxycarbonyloxy-C₁₋₈-alkyl,

C₁₋₈-alkoxy-C₃₋₈-cycloalkyl-C₁₋₈-alkyl

C₁₋₈-alkyl,

C₁₋₈-alkylsulfanyl,

C₁₋₈-alkylsulfanyl-C₁₋₈-alkoxy,

C₁₋₈-alkylsulfanyl-C₁₋₈-alkoxy-C₁₋₈-alkyl,

C₁₋₈-alkylsulfanyl-C₁₋₈-alkyl,

C₁₋₈-alkylsulfonyl-C₁₋₈-alkoxy-C₁₋₈-alkyl,

C₁₋₈-alkylsulfonyl-C₁₋₈-alkyl

C₂₋₈-alkynyl

optionally substituted C₁₋₈-alkoxy

optionally N-mono- or N,N-di-C₁₋₈-alkylated amino-C₁₋₈-alkoxy,

optionally N-mono- or N,N-di-C₁₋₈-alkylated amino-carbonyl-C₁₋₈-alkyl,

optionally substituted aryl-C₁₋₈-alkoxy-C₁₋₈-alkoxy,

optionally substituted aryl-heterocyclyl-C₀₋₈-alkoxy,

optionally substituted heterocyclyl-heterocyclyl-C₀₋₈-alkoxy,

optionally substituted aryl-C₀₋₈-alkoxy-C₁₋₈-alkoxy,

optionally substituted aryl-C₀₋₈-alkoxy-C₁₋₈-alkoxy-C₁₋₈-alkyl,

carboxy-C₁₋₈-alkyl,

cyano,

cyano-C₁₋₈-alkyl,

C₃₋₈-cycloalkyl-C₀₋₈-alkoxy-C₁₋₈-alkoxy,

C₃₋₈-cycloalkyl-C₀₋₈-alkoxy-C₁₋₈-alkoxy-C₁₋₈-alkyl

C₃₋₈-cycloalkyl-C₀₋₈-alkoxy-C₁₋₈-alkyl,

C₃₋₈-cycloalkyl-C₀₋₈-alkylamino-C₁₋₈-alkyl,

halogen-substituted C₁₋₈-alkoxy,

halogen-substituted C₁₋₈-alkyl,

halogen-substituted C₁₋₈-alkoxy-C₁₋₈-alkoxy-C₁₋₈-alkyl,

heterocyclyl-carbonyl-C₁₋₈-alkyl,

heterocyclyl-C₁₋₈-alkyl,

heterocyclyl-sulfanyl-C₁₋₈-alkoxy-C₁₋₈-alkyl and

heterocyclyl-C₀₋₈-alkoxy-C₁₋₈-alkyl;

and may, in addition to the aforementioned substituents, also be substituted by a maximum of 4 halogens;

R³ is

halogen- and/or hydroxy-substituted C₁₋₈-alkoxy,

optionally halogen- and/or hydroxy-substituted C₁₋₈-alkoxy-C₁₋₈-alkoxy,

optionally halogen- and/or hydroxy-substituted C₁₋₈-alkoxy-C₁₋₈-alkyl,

optionally N—C₁₋₈-alkylated C₁₋₈-alkoxy-C₁₋₈-alkylamino-C₁₋₈-alkoxy,

optionally N—C₁₋₈-alkylated C₁₋₈-alkoxy-C₁₋₈-alkylamino-C₁₋₈-alkyl,

C₁₋₈-alkoxy-C₀₋₈-alkylcarbonyl-C₀₋₈-alkoxy,

C₁₋₈-alkoxycarbonylamino-C₁₋₈-alkyl,

optionally halogen- and/or hydroxy-substituted C₁₋₈-alkyl,

optionally N—C₁₋₈-alkylated C₀₋₈-alkylcarbonylamino,

C₀₋₈-alkylcarbonylamino-C₁₋₈-alkoxy,

optionally N—C₁₋₈-alkylated and/or halogen-substituted C₀₋₈-alkylcarbonylamino-C₁₋₈-alkyl,

C₁₋₈-alkylcarbonyloxy

C₁₋₈-alkylsulfanyl-C₁₋₈-alkyl,

C₁₋₈-alkylsulfonyl-C₁₋₈-alkoxy,

C₁₋₈-alkylsulfonyl-C₁₋₈-alkyl,

C₂₋₈-alkynyloxy,

optionally N-mono- or N,N-di-C₁₋₈-alkylated amino-C₁₋₈-alkoxy,

optionally N-mono- or N,N-di-C₁₋₈-alkylated amino-C₁₋₈-alkyl,

optionally N-mono- or N,N-di-C₁₋₈-alkylated amino-C₀₋₈-alkylcarbonyl-C₁₋₈-alkoxy,

optionally N-mono- or N,N-di-C₁₋₈-alkylated amino-C₀₋₈-alkylcarbonyl-heterocyclyl-C₀₋₈-alkyl,

optionally N-mono- or N,N-di-C₁₋₈-alkylated amino-C₂₋₈-alkynyloxy,

optionally N-mono- or N,N-di-C₁₋₈-alkylated and optionally hydroxy-substituted amino-C₀₋₈-alkylcarbonyl-C₀₋₈-alkyl,

N-mono- or N,N-di-C₁₋₈-alkylated aminocarbonyl-C₂₋₈-alkynyloxy,

cyano,

cyano-C₁₋₈-alkoxy,

C₃₋₈-cycloalkyl-C₀₋₈-alkoxy,

optionally halogen-substituted C₃₋₈-cycloalkyl-C₀₋₈-alkylcarbonylamino-C₁₋₈-alkyl

C₃₋₈-cycloalkyl-carbonyloxy-C₀₋₈-alkyl

heterocyclyl-C₀₋₈-alkoxy,

heterocyclyl-C₀₋₈-alkyl,

optionally N—C₁₋₈-alkylated heterocyclyl-C₀₋₈-alkylamino-C₀₋₈-alkylcarbonyl-C₀₋₈-alkoxy,

optionally N—C₁₋₈-alkylated heterocyclyl-C₀₋₈-alkylamino-C₀₋₈-alkylcarbonyl-C₀₋₈-alkyl,

optionally halogen-substituted heterocyclyl-C₀₋₈-alkylcarbonylamino-C₁₋₈-alkyl,

heterocyclyl-C₂₋₈-alkynyloxy,

heterocyclylcarbonyl-C₀₋₈-alkoxy,

heterocyclylcarbonyl-C₀₋₈-alkyl,

heterocyclylcarbonyl-C₀₋₈-alkylamino-C₁₋₈-alkyl,

heterocyclyl-carbonyloxy-C₀₋₈-alkyl

optionally N—C₁₋₈-alkylated hydroxy-C₁₋₈-alkylamino-C₁₋₈-alkyl,

hydroxy-C₀₋₈-alkylcarbonyl-C₁₋₈-alkoxy or

optionally N—C₁₋₈-alkylated and/or halogen-substituted Cₒ₋₈-alkylcarbonylamino-C₁₋₈-alkoxy;
X is -Alk-, —O-Alk-, -Alk-O—, —O-Alk-O—, —S-Alk-, -Alk-S—, -Alk-NR⁴—, —NR⁴-Alk-, —C(O)—NR⁴—, -Alk-C(O)—NR⁴—, -Alk-C(O)—NR⁴-Alk-, —NR⁴—C(O)—, -Alk-NR⁴—C(O)—, —NR⁴—C(O)-Alk-, -Alk-NR⁴—C(O)-Alk-, —O-Alk-C(O)—NR⁴—, —O-Alk-NR⁴—C(O)—, —S(O)₂—NR⁴— or —S(O)₂—NR⁴-Alk-, where Alk is C₁₋₈-alkylene which may optionally be substituted by halogen; and where

R⁴ is hydrogen, C₁₋₈-alkyl, C₁₋₈-alkoxy-C₁₋₈-alkyl, acyl, C₃₋₈-cycloalkyl or aryl-C₁₋₈-alkyl; and the salts thereof, preferably the pharmaceutically acceptable salts thereof.

The linkage of the above (and hereinafter) mentioned substituent —X— within the compound of the formula (I) starts from the piperidine ring with the substituent —X— being arranged from left to right when written as indicated above. For example, the fragment “—X—R¹” of the compound of the formula (I) with X meaning “—NR⁴-Alk-” is: “—NR⁴-Alk-R¹”.

The meaning of “C₀-alkyl” in the above (and hereinafter) mentioned C₀₋₈-alkyl groups is a bond or, if located at a terminal position, a hydrogen atom.

The meaning of “C₀-alkoxy” in the above (and hereinafter) mentioned C₀₋₈-alkoxy groups is “—O—” or, if located at a terminal position, an —OH group.

C₁₋₈-Alkyl and alkoxy radicals may be linear or branched. Examples of C₁₋₈-alkyl and alkoxy radicals are methyl, ethyl, n-propyl, isopropyl, n-butyl, isobutyl, sec-butyl, tert-butyl, pentyl, hexyl, and methoxy, ethoxy, propoxy, isopropoxy, butoxy, isobutoxy, sec-butoxy and tert-butoxy. C₁₋₈-Alkylenedioxy radicals are preferably methylene-dioxy, ethylenedioxy and propylenedioxy. C₁₋₈-alkanoyl refers to C₁₋₈-alkylcarbonyl. Examples of C₁₋₈-alkanoyl radicals are acetyl, propionyl and butyryl.

As part of the substituent on R¹,
cycloalkyl refers to a saturated, cyclic hydrocarbon radical having 3 to 12 carbon atoms, for example cyclopropyl, cyclobutyl, cyclopentyl, cyclohexyl, cycloheptyl, bicyclo[2.2.1]heptyl, cyclooctyl, bicyclo[2.2.2]octyl and adamantyl, and may be unsubstituted or substituted one or more times, e.g. substituted once or twice by C₁₋₈-alkanoyl, C₂₋₈-alkenyl, C₂₋₈-alkynyl, C₁₋₈-alkoxy, C₁₋₈-alkoxy-C₁₋₈-alkoxy, C₁₋₈-alkoxy-C₁₋₈-alkyl, C₁₋₈-alkoxycarbonylamino, C₁₋₈-alkyl, C₀₋₈-alkylcarbonylamino, C₁₋₈-alkylcarbonyloxy, C₁₋₈-alkylenedioxy, optionally N-mono- or N,N-di-C₁₋₈-alkylated amino, aryl, optionally N-mono- or N,N-di-C₁₋₈-alkylated carbamoyl, optionally esterified carboxy, cyano, C₃₋₈-cycloalkoxy, halogen, heterocyclyl, hydroxy, oxo, halogen-substituted C₁₋₈-alkoxy or halogen-substituted C₁₋₈-alkyl.

As part of a substituent on R², as part of the substituent R³ or as R⁴,
cycloalkyl refers to a saturated cyclic hydrocarbon radicals having 3 to 8 carbon atoms, for example cyclopropyl, cyclobutyl or cyclopentyl and may be unsubstituted or substituted once or twice by C₁₋₈-alkoxy, C₁₋₈-alkoxy-C₁₋₈-alkyl, optionally halogen substituted C₁₋₈-alkyl or halogen.

Cycloalkyl radicals with two connection points may be linked via 2 different carbon atoms or via the same carbon atom, for example 1,1-cyclopropyl or 1,2-cyclopropyl.

C₁₋₈-Alkylene radicals may be linear or branched and are, for example, methylene, ethylene, propylene, 2-methylpropylene, 2-methylbutylene, 2-methylpropyl-2-ene, butyl-2-ene, butyl-3-ene, propyl-2-ene, tetra-, penta- and hexamethylene; C₂₋₈-alkenylene radicals are, for example, vinylene and propenylene; C₂₋₈-alkynylene radicals are, for example, ethynylene; acyl radicals are alkanoyl radicals, preferably C₁₋₈-alkanoyl radicals, or aroyl radicals such as benzoyl.

As R¹,
aryl refers to mono- or polynuclear aromatic radicals which may be substituted one or more times, such as, for example, phenyl, substituted phenyl, naphthyl, substituted naphthyl. Aryl refers also to bicyclic systems, where a monocyclic aryl radical has a 3-7-membered fused-on carbocyclic ring, such as, for example tetrahydronaphthyl or substituted tetrahydronaphthyl.

As part of a substituent on R¹ or R², or as part of the substituent R⁴,
aryl refers to mononuclear aromatic radicals which may be substituted one or more times, such as, for example, phenyl or substituted phenyl, and may be unsubstituted or substituted one or more times, e.g. substituted once or twice by C₁₋₈-alkoxy, C₁₋₈-alkyl, optionally esterified carboxy, cyano, halogen, hydroxy, halogen substituted C₁₋₈-alkoxy, halogen substituted C₁₋₈-alkyl or phenyl.

For R¹,
the term heterocyclyl refers to 3-16-membered, mono-, bi- or polycyclic, saturated, unsaturated and partially unsaturated heterocyclic radicals having 1 to 4 nitrogen and/or 1 or 2 sulfur or oxygen atoms. Preference is given to 3-8-membered, particularly preferably 5- or 6-membered, monocyclic radicals which optionally have a 3-8-membered fused-on ring, which may be carbocyclic or heterocyclic. A further preferred group of heterocyclic radicals are bi- or polycyclic heterocycles which optionally have a spirocyclic or bridged ring. Preferred heterocyclic radicals have in each ring 1 nitrogen, oxygen or sulfur atom, 1-2 nitrogen atoms and 1-2 oxygen atoms or 1-2 nitrogen atoms and 1-2 sulfur atoms, with at least one, preferably 1-7, carbon atoms being present in each ring. Heterocyclyl radicals may be substituted one or more times, in particular once, twice or three times.

Examples of unsaturated heterocyclyl radicals are
benzo[1,3]dioxolyl,
benzofuranyl,
benzoimidazolyl,
benzooxazolyl,
benzothiazolyl,
benzo[b]thienyl,
quinazolinyl,
quinolyl,
quinoxalinyl,
2H-chromenyl,
dihydrobenzofuranyl,
1,3-dihydrobenzoimidazolyl,
3,4-dihydro-2H-benzo[1,4]oxazinyl,
3,4-dihydro-3H-benzo[1,4]oxazinyl,
1,4-dihydrobenzo[d][1,3]oxazinyl,
3,4-dihydro-2H-benzo[1,4]thiazinyl,
3,4-dihydro-1 H-quinazolinyl,
3,4-dihydro-1H-quinolinyl,
2,3-dihydroindolyl,
2,3-dihydro-1H-pyrido[2,3-b][1,4]oxazinyl,
1,1-dioxodihydro-2H-benzo[1,4]thiazinyl,
furyl,
imidazolyl,
imidazo[1,5-a]pyridinyl,
imidazo[1,2-a]pyrimidinyl,
indazolyl,
indolyl,
isobenzofuranyl,
isoquinolyl,
[1,5]naphthyridyl,
oxazolyl,
phthalazinyl,
pyranyl,
pyrazinyl,
pyrazolyl,
pyridyl,
pyrimidinyl,
1H-pyrrolizinyl,
pyrrolo[3,2-c]pyridinyl,
pyrrolo[2,3-c]pyridinyl,
pyrrolo[3,2-b]pyridinyl,
1H-pyrrolo[2,3-b]pyridyl,
pyrrolyl,
1,3,4,5-tetrahydrobenzo[b]azepinyl,
tetrahydroquinolinyl,
tetrahydroquinoxalinyl,
tetrahydroisoquinolinyl,
thiazolyl,
thienyl,
triazinyl,
triazolyl.

Examples of saturated heterocyclyl radicals are
azepanyl,
azetidinyl,
aziridinyl,
3,4-dihydroxypyrrolidinyl,
2,6-dimethylmorpholinyl,
3,5-dimethylmorpholinyl,
dioxanyl,
[1,4]dioxepanyl,
dioxolanyl,
4,4-di-oxothiomorpholinyl,
dithianyl,
dithiolanyl,
2-hydroxymethylpyrrolidinyl,
4-hydroxypiperidinyl,
3-hydroxypyrrolidinyl,
4-methylpiperazinyl,
1-methylpiperidinyl,
1-methylpyrrolidinyl,
morpholinyl,
oxathianyl,
oxepanyl,
piperazinyl,
piperidinyl,
pyrrolidinyl,
tetrahydrofuranyl,
tetrahydropyranyl,
tetrahydrothiophenyl,
tetrahydrothiopyranyl,
thiepanyl,
thiomorpholinyl.

Examples of bi- or polycyclic saturated or partially unsaturated heterocyclyl radicals are
2,5-dioxabicyclo[4.1.0]heptanyl,
2-oxa-bicyclo[2.2.1]heptanyl,
2-oxabicyclo[4.1.0]heptanyl,
3-oxabicyclo[4.1.0]heptanyl,
7-oxa-bicyclo[2.2.1]heptanyl,
2-oxabicyclo[3.1.0]hexanyl,
3-oxabicyclo[3.1.0]hexanyl,
1-oxa-spiro[2.5]octanyl,
6-oxaspiro[2.5]octanyl, 3-oxabicyclo[3.3.1]nonanyl,
1a,7b-dihydro-1H-cyclopropa[c]chromenyl,
1,1a,2,7b-tetrahydrocyclopropa[c]chromenyl.

As part of a substituent on R¹ or as part of the substituent R³,
the term heterocyclyl refers to 3-7 membered monocyclic, saturated and unsaturated heterocyclic radicals having 1 to 4 nitrogen and/or 1 or 2 sulfur or oxygen atoms, which may be substituted one or more times, such as, for example, substituted once or twice by C₁₋₈-alkoxy, C₁₋₈-alkyl, optionally esterified carboxy, cyano, halogen, hydroxy, halogen-substituted C₁₋₈-alkoxy or halogen-substituted C₁₋₈-alkyl.
Examples of such heterocyclyl radicals are
imidazolyl,
morpholinyl,
oxetanyl,
oxiranyl,
pyrazolyl,
pyridyl,
pyrrolidinyl,
tetrahydrofuranyl,
tetrahydropyranyl,
tetrazolyl,
thiazolyl,
triazolyl.

As part of a substituent on R²,
the term heterocyclyl refers to 3-7 membered monocyclic, saturated, partially unsaturated and maximally unsaturated heterocyclic radicals having 1 to 5 nitrogen and/or 1 or 2 sulfur or oxygen atoms, which may be substituted one or more times, such as, for example, substituted once, twice or three times by C₁₋₈-alkoxy, C₁₋₈-alkoxy-C₁₋₈-alkyl, C₁₋₈-alkyl, aryl, cyano, halogen, heterocyclyl, hydroxy, halogen substituted C₁₋₈-alkoxy or halogen substituted C₁₋₈-alkyl.
Examples of such heterocycles are
imidazolyl,
oxetanyl,
pyrazolyl.
pyrrolidinyl,
tetrazolyl,
thiazolyl,
triazolyl.

Heterocyclyl radicals which comprise a nitrogen atom may be linked either via the N atom or via a C atom to the remainder of the molecule.

Hydroxy-substituted C₁₋₈-alkoxy may be for example hydroxy-C₁₋₈-alkoxy or else polyhydroxy-C₁₋₈-alkoxy.

The term halogen-substituted C₁₋₈-alkyl refers to C₁₋₈-alkyl radicals which may be substituted by 1-8 halogen atoms, such as, for example, bromo, chloro,fluoro, iodo. An analogous statement applies to radicals, such as halogen-substituted C₁₋₈-alkoxy.

The compounds of the formula (I) have at least two asymmetric carbon atoms and may therefore exist in the form of optically pure diastereomers, diastereomeric mixtures, diastereomeric racemates, mixtures of diastereomeric racemates or as meso compounds. The invention encompasses all these forms. Mixtures of diastereomers, diastereomeric racemates or mixtures of diastereomeric racemates can be fractionated by conventional methods, e.g. by column chromatography, thin-layer chromatography, HPLC and the like.

Salts are primarily the pharmaceutically acceptable or nontoxic salts of compounds of formula (I). The term “pharmaceutically acceptable salts” encompasses salts with inorganic or organic acids, such as hydrochloric acid, hydrobromic acid, nitric acid, sulfuric acid, phosphoric acid, citric acid, formic acid, maleic acid, acetic acid, succinic acid, tartaric acid, methanesulfonic acid, p-toluenesulfonic acid and the like.

Salts of compounds having salt-forming groups are in particular acid addition salts, salts with bases, or, in the presence of a plurality of salt-forming groups, in some cases also mixed salts or internal salts.

Such salts are formed, for example, from compounds of formula (I) with an acidic group, for example a carboxyl or sulfonyl group, and are, for example, the salts thereof with suitable bases such as non-toxic metal salts derived from metals of group Ia, Ib, IIa and IIb of the Periodic Table of the Elements, for example alkali metal, in particular lithium, sodium, or potassium, salts, alkaline earth metal salts, for example magnesium or calcium salts, and also zinc salts and ammonium salts, including those salts which are formed with organic amines, such as optionally hydroxy-substituted mono-, di- or trialkylamines, in particular mono-, di- or tri(lower alkyl)amines, or with quaternary ammonium bases, e.g. methyl-, ethyl-, diethyl- or triethylamine, mono-, bis- or tris(2-hydroxy(lower alkyl))amines, such as ethanol-, diethanol- or triethanolamine, tris(hydroxymethyl)methylamine or 2-hydroxy-tert-butylamine, N,N-di(lower alkyl)-N-(hydroxy(lower alkyl))amine, such as N,N-di-N-dimethyl-N-(2-hydroxyethyl)amine, or N-methyl-D-glucamine, or quaternary ammonium hydroxides such as tetrabutyl ammoniumhydroxide. The compounds of formula (I) having a basic group, for example an amino group, may form acid addition salts, for example with suitable inorganic acids, e.g. hydrohalic acid such as hydrochloric acid, hydrobromic acid, sulfuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulfonic or phosphonic acids or N-substituted sulfamic acids, e.g. acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, and also amino acids, for example the alpha-amino acids mentioned above, and also methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, 2- or 3-phosphoglycerate, glucose 6-phosphate, N-cyclohexylsulfamic acid (with formation of the cyclamates) or with other acidic organic compounds such as ascorbic acid. Compounds of formula (I) having acidic and basic groups may also form internal salts.

Salts obtained may be converted to other salts in a manner known per se, acid addition salts, for example, by treating with a suitable metal salt such as a sodium, barium or silver salt, of another acid in a suitable solvent in which an inorganic salt which forms is insoluble and thus separates out of the reaction equilibrium, and base salts by release of the free acid and salt reformation.

The compounds of formula (I), including their salts, may also be obtained in the form of hydrates or include the solvent used for the crystallization.

For the isolation and purification, pharmaceutically unsuitable salts may also find use.

The compound groups mentioned below are not to be regarded as closed, but rather parts of these compound groups may be exchanged with one another or with the definitions given above or omitted in a sensible manner, for example to replace general by more specific definitions. The definitions are valid in accordance with general chemical principles, such as, for example, the common valences for atoms.

Preferred compounds according to the invention are those of the general formula (IA) and the salts thereof, preferably the pharmaceutically acceptable salts thereof.

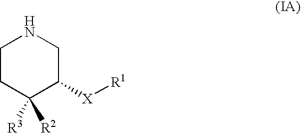

in which R¹, R², R³, and X have the meaning indicated above for the compounds of the formula (I).

A further preferred group of compounds of the formula (I), and particularly preferably of the formula (IA), and the salts thereof, preferably the pharmaceutically acceptable salts thereof, are compounds in which

R¹ is phenyl or heterocyclyl, each substituted as indicated above for compounds of the formula (I).

Particularly preferred heterocyclic R¹ radicals are

benzo[1,3]dioxolyl,

benzofuranyl,

benzoimidazolyl,

4H-benzo[1,4]oxazinyl,

benzooxazolyl,

4H-benzo[1,4]thiazinyl,

quinolinyl,

2H-chromenyl,

dihydro-benzo[e][1,4]diazepinyl,

3,4-dihydro-2H-benzo[1,4]oxazinyl,

3,4-dihydro-3H-benzo[1,4]oxazinyl,

1,4-dihydro-2H-benzo[d][1,3]oxazinyl,

3,4-dihydro-2H-benzo[1,4]thiazinyl,

1a,7b-dihydro-1H-cyclopropa[c]chromenyl,

1,3-dihydroindolyl,

2,3-dihydroindolyl,

2,3-dihydro-1H-pyrido[2,3-b][1,4]oxazinyl,

imidazo[1,5-a]pyridinyl,

indazolyl,

indolyl,

3H-isobenzofuranyl,

[1,5]naphthyridyl,

oxazolyl,

phthalazinyl,

pyrazolyl,

1H-pyrido[2,3-b][1,4]oxazinyl,

pyridyl,

pyrimidinyl

1H-pyrrolizinyl,

1H-pyrrolo[2,3-b]pyridyl,

pyrrolyl,

tetrahydrobenzo[e][1,4]diazepinyl,

2H-thieno[2,3-d]pyrimidinyl,

tetrahydro-quinoxalinyl,

1,1a,2,7b-tetrahydrocyclopropa[c]chromenyl and

triazinyl.

Particularly preferred radicals R¹ are

benzo[1,3]dioxolyl,

benzofuranyl,

benzoimidazolyl,

4H-benzo[1,4]oxazinyl,

benzooxazolyl,

4H-benzo[1,4]thiazinyl,

2H-chromenyl,

dihydro-benzo[e][1,4]diazepinyl,

3,4-dihydro-2H-benzo[1,4]oxazinyl,

3,4-dihydro-3H-benzo[1,4]oxazinyl,

1,4-dihydro-2H-benzo[d][1,3]oxazinyl,

3,4-dihydro-2H-benzo[1,4]thiazinyl,

1a,7b-dihydro-1 H-cyclopropa[c]chromenyl,

1,3-dihydroindolyl,

2,3-dihydroindolyl,

2,3-dihydro-1H-pyrido[2,3-b][1,4]oxazinyl,

imidazo[1,5-a]pyridinyl,

indazolyl,

indolyl,

3H-isobenzofuranyl,

1H-pyrido[2,3-b][1,4]oxazinyl,

phenyl,

pyridyl,

pyrimidinyl

1H-pyrrolo[2,3-b]pyridyl,

1,1a,2,7b-tetrahydrocyclopropa[c]chromenyl and

triazinyl;

substituted by 1-3 radicals independently selected from the group consisting of

C₁₋₈-alkanoyl,

C₁₋₈-alkoxy,

C₁₋₈-alkoxy-C₁₋₈-alkoxy,

C₁₋₈-alkoxy-C₁₋₈-alkoxy-C₁₋₈-alkyl,

C₁₋₈-alkoxy-C₁₋₈-alkyl,

(N—C₁₋₈-alkoxy)-C₁₋₈-alkylaminocarbonyl-C₁₋₈-alkoxy,

(N—C₁₋₈-alkoxy)-C₁₋₈-alkylaminocarbonyl-C₁₋₈-alkyl,

C₁₋₈-alkoxy-C₁₋₈-alkylcarbonyl,

C₁₋₈-alkoxycarbonylamino-C₁₋₈-alkoxy,

C₁₋₈-alkoxycarbonylamino-C₁₋₈-alkyl,

C₁₋₈-alkyl,

(N—C₁₋₈-alkyl)-C₀₋₈-alkylcarbonylamino-C₁₋₈-alkoxy,

(N—C₁₋₈-alkyl)-C₀₋₈-alkylcarbonylamino-C₁₋₈-alkyl,

C₀₋₈-alkylcarbonylamino-C₁₋₈-alkoxy,

C₀₋₈-alkylcarbonylamino-C₁₋₈-alkyl,

halogen,

oxide,

oxo,

halogen substituted C₁₋₈-alkoxy,

halogen substituted C₁₋₈-alkyl,

heterocyclyl-C₁₋₈-alkoxy and

heterocyclyl-C₁₋₈-alkyl.

R¹ is very particularly preferably

2H-chromenyl,

3,4-dihydro-2H-benzo[1,4]oxazinyl,

3,4-dihydro-2H-benzo[1,4]thiazinyl or

1,3-dihydroindolyl

substituted by 1-3 radicals independently selected from the group consisting of

C₁₋₈-alkoxy,

C₁₋₈-alkoxy-C₁₋₈-alkoxy,

C₁₋₈-alkoxy-C₁₋₈-alkoxy-C₁₋₈-alkyl,

C₁₋₈-alkoxy-C₁₋₈-alkyl,

C₁₋₈-alkoxy-C₁₋₈-alkylcarbonyl,

C₁₋₈-alkoxycarbonylamino-C₁₋₈-alkoxy,

C₁₋₈-alkoxycarbonylamino-C₁₋₈-alkyl,

C₁₋₈-alkyl,

(N—C₁₋₈-alkyl)-C₀₋₈-alkylcarbonylamino-C₁₋₈-alkoxy,

(N—C₁₋₈-alkyl)-C₀₋₈-alkylcarbonylamino-C₁₋₈-alkyl,

C₀₋₈-alkylcarbonylamino-C₁₋₈-alkoxy,

C₀₋₈-alkylcarbonylamino-C₁₋₈-alkyl,

halogen,

oxo,

halogen-substituted C₁₋₈-alkoxy and

halogen-substituted C₁₋₈-alkyl.

Preference is furthermore given to compounds of the formulae (I) and (IA) and the salts thereof, preferably the pharmaceutically acceptable salts thereof, in which R³ is

halogen- and/or hydroxy-substituted C₁₋₈-alkoxy,

optionally halogen- and/or hydroxy-substituted C₁₋₈-alkoxy-C₁₋₈-alkoxy,

optionally halogen- and/or hydroxy-substituted C₁₋₈-alkoxy-C₁₋₈-alkyl,

optionally halogen- and/or hydroxy-substituted C₁₋₈-alkyl,

optionally N—C₁₋₈-alkylated C₁₋₈-alkylcarbonylamino,

optionally N—C₁₋₈-alkylated and/or halogen-substituted C₀₋₈-alkylcarbonylamino-C₁₋₈-alkyl, C₁₋₈-alkylcarbonyloxy

optionally N-mono- or N,N-di-C₁₋₈-alkylated amino-C₀₋₈-alkylcarbonyl-C₁₋₈-alkoxy,

optionally N-mono- or N,N-di-C₁₋₈-alkylated amino-C₀₋₈-alkylcarbonyl-heterocyclyl-C₀₋₈-alkyl,

heterocyclyl-C₀₋₈-alkyl,

optionally N-mono- or N,N-di-C₁₋₈-alkylated and optionally hydroxy-substituted amino-C₀₋₈alkylcarbonyl-Cₒ₋₈-alkyl,

cyano,

cyano-C₁₋₈-alkoxy,

C₃₋₈-cycloalkyl-C₀₋₈-alkoxy,

C₃₋₈-cycloalkyl-carbonyloxy-C₀₋₈-alkyl

heterocyclyl-C₀₋₈-alkoxy,

heterocyclylcarbonyl-C₀₋₈-alkyl or

optionally N—C₁₋₈-alkylated and/or halogen-substituted C₀₋₈-alkylcarbonylamino-C₁₋₈-alkoxy.

R³ is very particularly preferably

halogen- and/or hydroxy-substituted C₁₋₈-alkoxy,

optionally halogen- and/or hydroxy-substituted C₁₋₈-alkoxy-C₁₋₈-alkoxy,

optionally halogen- and/or hydroxy-substituted C₁₋₈-alkoxy-C₁₋₈-alkyl,

optionally halogen- and/or hydroxy-substituted C₁₋₈-alkyl,

optionally N—C₁₋₈-alkylated C₁₋₈-alkylcarbonylamino,

optionally N—C₁₋₈-alkylated and/or halogen-substituted C₀₋₈-alkylcarbonylamino-C₁₋₈-alkyl,

C₁₋₈-alkylcarbonyloxy

optionally N-mono- or N,N-di-C₁₋₈-alkylated amino-C₀₋₈-alkylcarbonyl-C₁₋₈-alkoxy,

cyano,

cyano-C₁₋₈-alkoxy,

C₃₋₈-cycloalkyl-C₀₋₈-alkoxy,

C₃₋₈-cycloalkyl-carbonyloxy-C₀₋₈-alkyl

heterocyclyl-C₀₋₈-alkoxy or

optionally N—C₁₋₈-alkylated and/or halogen-substituted C₀₋₈-alkylcarbonylamino-C₁₋₈-alkoxy.

Preference is furthermore given to compounds of the formulae (I) and (IA) and the salts thereof, preferably the pharmaceutically acceptable salts thereof, in which R² is phenyl, substituted as indicated above for compounds of the formula (I) and in which R¹, R³, and X have the meaning indicated above for the compounds of the formula (I).

Preference is furthermore given to compounds of the formulae (I) and (IA) and the salts thereof, preferably the pharmaceutically acceptable salts thereof, in which R² is pyridyl, substituted as indicated above for compounds of the formula (I) and in which R¹, R³, and X have the meaning indicated above for the compounds of the formula (I).

Preference is furthermore given to compounds of the formulae (I) and (IA) and the salts thereof, preferably the pharmaceutically acceptable salts thereof, in which R² is phenyl or pyridyl, where the nitrogen atom of the pyridyl is located in the ortho- or meta-position relative to the bond from the pyridyl ring to the remainder of the molecule and where the phenyl or pyridyl is substituted by 1-3 radicals, preferably one of which is located in the para-position relative to the bond from the phenyl or pyridyl ring to the remainder of the molecule, independently selected from the group consisting of

C₁₋₈-alkoxy,

C₁₋₈-alkoxy-C₁₋₈-alkoxy,

C₁₋₈-alkoxy-C₁₋₈-alkoxy-C₁₋₈-alkoxy,

C₁₋₈-alkoxy-C₁₋₈-alkoxy-C₁₋₈-alkoxy-C₁₋₈-alkyl,

C₁₋₈-alkoxy-C₁₋₈-alkoxy-C₁₋₈-alkyl,

C₁₋₈-alkoxy-C₁₋₈-alkyl,

C₁₋₈-alkoxy-C₀₋₈-alkyl-C₃₋₈-cycloalkyl-C₀₋₈-alkoxy-C₁₋₈-alkyl,

C₁₋₈-alkoxy-C₁₋₈-alkylsulfanyl,

C₁₋₈-alkoxy-C₁₋₈-alkylsulfanyl-C₁₋₈-alkyl,

C₁₋₈-alkoxy-C₃₋₈-cycloalkyl-C₁₋₈-alkyl

C₁₋₈-alkyl,

C₁₋₈-alkylsulfanyl-C₁₋₈-alkoxy,

C₁₋₈-alkylsulfanyl-C₁₋₈-alkoxy-C₁₋₈-alkyl,

optionally substituted C₁₋₈-alkoxy

optionally substituted aryl-heterocyclyl-C₀₋₈-alkoxy,

C₃₋₈-cycloalkyl-C₀₋₈-alkoxy-C₁₋₈-alkyl,

halogen-substituted C₁₋₈-alkoxy,

halogen-substituted C₁₋₈-alkyl,

heterocyclyl-C₀₋₈-alkoxy-C₁₋₈-alkyl and

optionally substituted heterocyclyl-heterocyclyl-C₀₋₈-alkoxy.

R² is particularly preferably

phenyl or pyridyl, where the nitrogen atom of the pyridyl is located in the ortho- or meta-position relative to the bond from the pyridyl ring to the remainder of the molecule and where the phenyl or pyridyl is substituted by 1-2 radicals, preferably one of which is located in the para-position relative to the bond from the phenyl or pyridyl ring to the remainder of the molecule, independently selected from the group consisting of

C₁₋₈-alkoxy,

C₁₋₈-alkoxy-C₁₋₈-alkoxy,

C₁₋₈-alkoxy-C₁₋₈-alkoxy-C₁₋₈-alkoxy,

C₁₋₈-alkoxy-C₁₋₈-alkoxy-C₁₋₈-alkoxy-C₁₋₈-alkyl,

C₁₋₈-alkoxy-C₁₋₈-alkoxy-C₁₋₈-alkyl,

optionally substituted C₁₋₈-alkoxy

C₁₋₈-alkyl,

C₃₋₈-cycloalkyl-C₀₋₈-alkoxy-C₁₋₈-alkyl,

heterocyclyl-C₀₋₈-alkoxy-C₁₋₈-alkyl,

optionally substituted aryl-heterocyclyl-C₀₋₈-alkoxy and

optionally substituted heterocyclyl-pyrrolidinyl-C₀₋₈-alkoxy;

R² is very particularly preferably

phenyl, preferably para-substituted, relative to the bond from the phenyl ring to the remainder of the molecule, by one radical selected from the group consisting of

C₁₋₈-alkoxy,

C₁₋₈-alkoxy-C₁₋₈-alkoxy,

C₁₋₈-alkoxy-C₁₋₈-alkoxy-C₁₋₈-alkoxy,

C₁₋₈-alkoxy-C₁₋₈-alkoxy-C₁₋₈-alkoxy-C₁₋₈-alkyl,

C₁₋₈-alkoxy-C₁₋₈-alkoxy-C₁₋₈-alkyl,

optionally substituted C₁₋₈-alkoxy

C₁₋₈-alkyl,

C₃₋₈-cycloalkyl-C₀₋₈-alkoxy-C₁₋₈-alkyl,

heterocyclyl-C₀₋₈-alkoxy-C₁₋₈-alkyl,

optionally substituted aryl-heterocyclyl-C₀₋₈-alkoxy and

optionally substituted heterocyclyl-pyrrolidinyl-C₀₋₈-alkoxy.

A further preferred group of compounds of the formula (I), and particularly preferably of the formula (IA), and the salts thereof, preferably the pharmaceutically acceptable salts thereof, are compounds in which

X is -Alk-, —O-Alk- or —O-Alk-O— where Alk is C₁₋₈-alkylene.

X is particularly preferred —O-Alk-, and very particularly preferred —O—CH₂—.

Preference is furthermore given to compounds of the formulae (I) and (IA) and the salts thereof, preferably the pharmaceutically acceptable salts thereof, in which

R² is

phenyl or pyridyl, where the nitrogen atom of the pyridyl is located in the ortho- or meta-position relative to the bond from the pyridyl ring to the remainder of the molecule and where the phenyl or pyridyl is substituted by one radical, preferably located in the para-position relative to the bond from the phenyl or pyridyl ring to the remainder of the molecule, selected from the group consisting of

C₁₋₈-alkoxy,

C₁₋₈-alkoxy-C₁₋₈-alkoxy,

C₁₋₈-alkoxy-C₁₋₈-alkoxy-C₁₋₈-alkoxy,

C₁₋₈-alkoxy-C₁₋₈-alkoxy-C₁₋₈-alkoxy-C₁₋₈-alkyl,

C₁₋₈-alkoxy-C₁₋₈-alkoxy-C₁₋₈-alkyl,

optionally substituted C₁₋₈-alkoxy,

C₁₋₈-alkyl,

C₃₋₈-cycloalkyl-C₀₋₈-alkoxy-C₁₋₈-alkyl,

heterocyclyl-C₀₋₈-alkoxy-C₁₋₈-alkyl,

optionally substituted aryl-heterocyclyl-C₀₋₈-alkoxy and

optionally substituted heterocyclyl-pyrrolidinyl-C₀₋₈-alkoxy;

and

R³ is

halogen- and/or hydroxy-substituted C₁₋₈-alkoxy,

optionally halogen- and/or hydroxy-substituted C₁₋₈-alkoxy-C₁₋₈-alkoxy,

optionally halogen- or hydroxy-substituted C₁₋₈-alkoxy-C₁₋₈-alkyl,

optionally halogen- and/or hydroxy-substituted C₁₋₈-alkyl,

optionally N—C₁₋₈-alkylated C₁₋₈-alkylcarbonylamino,

optionally N—C₁₋₈-alkylated and/or halogen-substituted C₀₋₈-alkylcarbonylamino-C₁₋₈-alkyl,

C₁₋₈-alkylcarbonyloxy

optionally N-mono- or N,N-di-C₁₋₈-alkylated amino-C₀₋₈-alkylcarbonyl-C₁₋₈-alkoxy,

cyano,

cyano-C₁₋₈-alkoxy,

C₃₋₈-cycloalkyl-C₀₋₈-alkoxy,

C₃₋₈-cycloalkyl-carbonyloxy-C₀₋₈-alkyl

heterocyclyl-C₀₋₈-alkoxy or

optionally N—C₁₋₈-alkylated and/or halogen-substituted C₀₋₈-alkylcarbonylamino-C₁₋₈-alkoxy,

Very particular preference is given to compounds and the salts thereof, preferably the pharmaceutically acceptable salts thereof, of the formulae (I) and (IA) in which

R¹ is 2H-chromenyl or 3,4-dihydro-2H-benzo[1,4]oxazinyl, substituted as defined for compounds of formula (I);

R² is

phenyl, preferably para-substituted, relative to the bond from the phenyl ring to the remainder of the molecule, by one radical selected from the group consisting of

C₁₋₈-alkoxy,

C₁₋₈-alkoxy-C₁₋₈-alkoxy,

C₁₋₈-alkoxy-C₁₋₈-alkoxy-C₁₋₈-alkoxy,

C₁₋₈-alkoxy-C₁₋₈-alkoxy-C₁₋₈-alkyl,

C₁₋₈-alkyl,

C₃₋₈-cycloalkyl-C₀₋₈-alkoxy-C₁₋₈-alkyl,

heterocyclyl-C₀₋₈-alkoxy-C₁₋₈-alkyl,

optionally substituted aryl-pyrrolidinyl-C₀₋₈-alkoxy and

optionally substituted heterocyclyl-pyrrolidinyl-C₀₋₈-alkoxy;

R³ is

halogen- and/or hydroxy-substituted C₁₋₈-alkoxy,

optionally halogen- and/or hydroxy-substituted C₁₋₈-alkoxy-C₁₋₈-alkoxy,

optionally halogen- or hydroxy-substituted C₁₋₈-alkoxy-C₁₋₈-alkyl,

optionally halogen- and/or hydroxy-substituted C₁₋₈-alkyl,

optionally N—C₁₋₈-alkylated C₁₋₈-alkylcarbonylamino,

optionally N—C₁₋₈-alkylated and/or halogen-substituted C₀₋₈-alkylcarbonylamino-C₁₋₈-alkyl,

C₁₋₈-alkylcarbonyloxy

optionally N-mono- or N,N-di-C₁₋₈-alkylated amino-C₀₋₈-alkylcarbonyl-C₁₋₈-alkoxy,

cyano,

cyano-C₁₋₈-alkoxy,

C₃₋₈-cycloalkyl-C₀₋₈-alkoxy,

C₃₋₈-cycloalkyl-carbonyloxy-C₀₋₈-alkyl

heterocyclyl-C₀₋₈-alkoxy or

optionally N—C₁₋₈-alkylated and/or halogen-substituted C₀₋₈-alkylcarbonylamino-C₁₋₈-alkoxy;

and

X is -Alk-, —O-Alk- or —O-Alk-O— where Alk is C₁₋₈-alkylene.

The compounds of the formulae (I) and (IA) can be prepared in an analogous manner to preparation processes disclosed in the literature. Similar preparation processes are described for example in WO 97/09311 and WO 00/063173. Details of the specific preparation variants can be found in the examples.

The compounds of the present invention are, from a synthetical point of view, easily accessible and can be prepared in a reasonable amount of steps. Especially compared to structurally related compounds known from WO 2006/103275, the synthetic effort is much reduced, since the compounds of the present invention contain one less stereocenter.

The compounds of the formula (I) can also be prepared in optically pure form. Separation into antipodes can take place by methods known per se, either preferably at an early stage in the synthesis by salt formation with an optically active acid such as, for example, (+)- or (−)-mandelic acid and separation of the diastereomeric salts by fractional crystallization or preferably at a rather late stage by derivatizing with a chiral auxiliary component such as, for example, (+)- or (−)-camphanoyl chloride, and separation of the diastereomeric products by chromatography and/or crystallization and subsequent cleavage of the linkage to the chiral auxiliary. The pure diastereomeric salts and derivatives can be analysed to determine the absolute configuration of the contained piperidine by conventional spectroscopic methods, with X-ray spectroscopy on single crystals representing a particularly suitable method.

It is possible for the configuration at individual chiral centres in a compound of formula (I) to be inverted selectively. For example, the configuration of asymmetric carbon atoms which bear nucleophilic substituents, such as amino or hydroxyl, may be inverted by second-order nucleophilic substitution, if appropriate after conversion of the bonded nucleophilic substituent to a suitable nucleofugic leaving group and reaction with a reagent which introduces the original substituents, or the configuration at carbon atoms having hydroxyl groups can be inverted by oxidation and reduction, analogously to the process in the European patent application EP-A-0 236 734. Also advantageous is the reactive functional modification of the hydroxyl group and subsequent replacement thereof by hydroxyl with inversion of configuration.

The compounds of the formula (I) and (IA) also include compounds in which one or more atoms are replaced by their stable, non-radioactive isotopes; for example a hydrogen atom by deuterium.

The compounds of the formula (I) also include compounds that have been nitrosated through one or more sites such as oxygen (hydroxyl condensation), sulfur (sulfhydryl condensation) and/or nitrogen. The nitrosated compounds of the present invention can be prepared using conventional methods known to one skilled in the art. For example, known methods for nitrosating compounds are described in WO 2004/098538 A2.

The compounds of the formula (I) also include compounds that have been converted at one or more sites such that a nitrate-ester-containing linker is attached to an existing oxygen and/or nitrogen. Such “nitroderivatives” of the compounds of the present invention can be prepared using conventional methods known to one skilled in the art. For example, known methods for converting compounds into their nitroderivatives are described in WO 2007/045551 A2.

Prodrug derivatives of the compounds described herein are derivatives thereof which on in vivo use liberate the original compound by a chemical or physiological process. A prodrug may for example be converted into the original compound when a physiological pH is reached or by enzymatic conversion. Possible examples of prodrug derivatives are esters of freely available carboxylic acids, S- and O-acyl derivatives of thiols, alcohols or phenols, the acyl group being defined as above. Preferred derivatives are pharmaceutically acceptable ester derivatives which are converted by solvolysis in physiological medium into the original carboxylic acid, such as, for example, lower alkyl esters, cycloalkyl esters, lower alkenyl esters, benzyl esters, mono- or disubstituted lower alkyl esters such as lower ω-(amino, mono- or dialkylamino, carboxy, lower alkoxycarbonyl)—alkyl esters or such as lower α-(alkanoyloxy, alkoxycarbonyl or dialkylaminocarbonyl)—alkyl esters; conventionally, pivaloyloxymethyl esters and similar esters are used as such.

Because of the close relationship between a free compound, a prodrug derivative and a salt compound, a particular compound in this invention also includes its prodrug derivative and salt form, where this is possible and appropriate.

The compounds of the formula (I), and preferably of the formula (IA), and their pharmaceutically acceptable salts have an inhibitory effect on the natural enzyme renin. The latter passes from the kidneys into the blood and there brings about the cleavage of angiotensinogen to form the decapeptide angiotensin I which is then cleaved in the lung, the kidneys and other organs to the octapeptide angiotensin II. Angiotensin II raises the blood pressure both directly by arterial constriction, and indirectly by releasing the hormone aldosterone, which retains sodium ions, from the adrenals, which is associated with an increase in the extracellular fluid volume. This increase is attributable to the effect of angiotensin II itself or of the heptapeptide angiotensin III formed therefrom as cleavage product. Inhibitors of the enzymatic activity of renin bring about a reduction in the formation of angiotensin I and, as a consequence thereof, the formation of a smaller amount of angiotensin II. The reduced concentration of this active peptide hormone is the direct cause of the blood pressure-lowering effect of renin inhibitors.

The effect of renin inhibitors is detected inter alia experimentally by means of in vitro tests where the reduction in the formation of angiotensin I is measured in various systems (human plasma, purified human renin together with synthetic or natural renin substrate). The following in vitro test of Nussberger et al. (1987) J. Cardiovascular Pharmacol., Vol. 9, pp. 39-44, is used inter alia. This test measures the formation of angiotensin I in human plasma. The amount of angiotensin I formed is determined in a subsequent radioimmunoassay. The effect of inhibitors on the formation of angiotensin I is tested in this system by adding various concentrations of these substances. The IC₅₀ is defined as the concentration of the particular inhibitor which reduces the formation of angiotensin I by 50%. The compounds of the present invention show inhibitory effects in the in vitro systems at minimal concentrations of about 10⁻⁶ to about 10⁻¹⁰ mol/l.

Illustrative of the invention, the following IC₅₀ values are given:

| Example No. | IC₅₀ (nM)* |
|---|---|
| 1 | 26.5 |
| 2 | 10.4 |
| 3 | 9.5 |
| 5 | 92.5 |
| 10 | 3.0 |
| 12 | 7.5 |

*A lower inhibiting activity corresponds to a higher IC₅₀ value

Renin inhibitors bring about a fall in blood pressure in salt-depleted animals. Human renin differs from renin of other species. Inhibitors of human renin are tested using primates (marmosets, Callithrix jacchus) because human renin and primate renin are substantially homologous in the enzymatically active region. The following in vivo test is employed inter alia: the test compounds are tested on normotensive marmosets of both sexes with a body weight of about 350 g, which are conscious, unrestrained and in their normal cages. Blood pressure and heart rate are measured with a catheter in the descending aorta and are recorded radiometrically. Endogenous release of renin is stimulated by combining a low-salt diet for 1 week with a single intramuscular injection of furosemide (5-(aminosulfonyl)-4-chloro-2-[(2-furanylmethyl)amino]benzoic acid) (5 mg/kg). 16 hours after the furosemide injection, the test substances are administered either directly into the femoral artery by means of a hypodermic needle or as suspension or solution by gavage into the stomach, and their effect on blood pressure and heart rate is evaluated. The compounds of the present invention have a blood pressure-lowering effect in the described in vivo test with i.v. doses of about 0.003 to about 0.3 mg/kg and with oral doses of about 0.3 to about 30 mg/kg.

The blood pressure-reducing effect of the compounds described herein can be tested in vivo using the following protocol:

The investigations take place in 5 to 6-week old, male double transgenic rats (dTGR), which overexpress both human angiotensinogen and human renin and consequently develop hypertension (Bohlender J. et al., J. Am. Soc. Nephrol. 2000; 11: 2056-2061). This double transgenic rat strain was produced by crossbreeding two transgenic strains, one for human angiotensinogen with the endogenous promoter and one for human renin with the endogenous promoter. Neither single transgenic strain was hypertensive. The double transgenic rats, both males and females, develop severe hypertension (mean systolic pressure, approximately 200 mm Hg) and die after a median of 55 days if untreated. The fact that human renin can be studied in the rat is a unique feature of this model. Age-matched Sprague-Dawley rats serve as non-hypertensive control animals. The animals are divided into treatment groups and receive test substance or vehicle (control) for various treatment durations. The applied doses for oral administration may range from 0.5 to 100 mg/kg body weight. Throughout the study, the animals receive standard feed and tap water ad libitum. The systolic and diastolic blood pressure, and the heart rate are measured telemetrically by means of transducers implanted in the abdominal aorta, allowing the animals free and unrestricted movement.

The effect of the compounds described herein on kidney damage (proteinuria) can be tested in vivo using the following protocol:

The investigations take place in 4-week old, male double transgenic rats (dTGR), as described above. The animals are divided into treatment groups and receive test substance or vehicle (control) each day for 7 weeks. The applied doses for oral administration may range from 0.5 to 100 mg/kg body weight. Throughout the study, the animals receive standard feed and tap water ad libitum. The animals are placed periodically in metabolism cages in order to determine the 24-hour urinary excretion of albumin, diuresis, natriuresis, and urine osmolality. At the end of the study, the animals are sacrificed and the kidneys and hearts may also be removed for determining the weight and for immunohistological investigations (fibrosis, macrophage/T cell infiltration, etc.).

The pharmacokinetic properties of the compounds described herein can be tested in vivo using the following protocol:

The investigations take place in pre-catheterized (carotid artery) male rats (300 g±20%) that can move freely throughout the study. The compound is administered intravenously and orally (gavage) in separate sets of animals. The applied doses for oral administration may range from 0.5 to 50 mg/kg body weight; the doses for intravenous administration may range from 0.5 to 20 mg/kg body weight. Blood samples are collected through the catheter before compound administration and over the subsequent 24-hour period using an automated sampling device (AccuSampler, DiLab Europe, Lund, Sweden). Plasma levels of the compound are determined using a validated LC-MS analytical method. The pharmacokinetic analysis is performed on the plasma concentration-time curves after averaging all plasma concentrations across time points for each route of administration. Typical pharmacokinetics parameters to be calculated include: maximum concentration (Cₘₐₓ), time to maximum concentration (tₘₐₓ), area under the curve from 0 hours to the time point of the last quantifiable concentration (AUC₀₋ₜ), area under the curve from time 0 to infinity (AUC_{0-inf}), elimination rate constant (K), terminal half-life (t_{1/2}), absolute oral bioavailability or fraction absorbed (F), clearance (CL), and Volume of distribution during the terminal phase (Vd).

The compounds of the formula (I), and preferably of the formula (IA), and their pharmaceutically acceptable salts can be used as medicines, e.g. in the form of pharmaceutical products. The pharmaceutical products can be administered enterally, such as orally, e.g. in the form of tablets, lacquered tablets, sugar-coated tablets, hard and soft gelatine capsules, solutions, emulsions or suspensions, nasally, e.g. in the form of nasal sprays, rectally, e.g. in the form of suppositories, or transdermally, e.g. in the form of ointments or patches. However, administration is also possible parenterally, such as intramuscularly or intravenously, e.g. in the form of solutions for injection.

Tablets, lacquered tablets, sugar-coated tablets and hard gelatine capsules can be produced by processing the compounds of the formula (I), or preferably of the formula (IA), and their pharmaceutically acceptable salts with pharmaceutically inert inorganic or organic excipients. Excipients of these types which can be used for example for tablets, sugar-coated tablets and hard gelatine capsules are lactose, maize starch or derivatives thereof, talc, stearic acid or salts thereof etc.

Excipients suitable for soft gelatine capsules are, for example, vegetable oils, waxes, fats, semisolid and liquid polyols etc.

Excipients suitable for producing solutions and syrups are, for example, water, polyols, sucrose, invert sugar, glucose etc.

Excipients suitable for solutions for injection are, for example, water, alcohols, polyols, glycerol, vegetable oils, bile acids, lecithin etc.

Excipients suitable for suppositories are, for example, natural or hardened oils, waxes, fats, semiliquid or liquid polyols etc.

The pharmaceutical products may in addition comprise preservatives, solubilizers, viscosity-increasing substances, stabilizers, wetting agents, emulsifiers, sweeteners, colorants, aromatizers, salts to alter the osmotic pressure, buffers, coating agents or antioxidants. They may also comprise other substances of therapeutic value.

The present invention further provides the use of the compounds of the formula (I), or preferably of the formula (IA), and their pharmaceutically acceptable salts in the treatment or prevention of high blood pressure, heart failure, glaucoma, myocardial infarction, renal failure, restenoses and stroke.

The present invention further provides the use of a compound of the general formula (I) or (IA) or a pharmaceutically acceptable salt thereof, for producing a human medicine for preventing, for delaying the progression of or for treating high blood pressure, heart failure, glaucoma, myocardial infarction, renal failure, restenoses or stroke.

The compounds of the formula (I), and preferably of the formula (IA), and their pharmaceutically acceptable salts can also be administered in combination with one or more agents having cardiovascular activity, e.g. α- and β-blockers such as phentolamine, phenoxybenzamine, prazosin, terazosin, tolazine, atenolol, metoprolol, nadolol, propranolol, timolol, carteolol etc.; vasodilators such as hydralazine, minoxidil, diazoxide, nitroprusside, flosequinan etc.; calcium antagonists such as amrinone, bencyclan, diltiazem, fendiline, flunarizine, nicardipine, nimodipine, perhexiline, verapamil, gallopamil, nifedipine etc.; ACE inhibitors such as cilazapril, captopril, enalapril, lisinopril etc.; potassium activators such as pinacidil; antiserotoninergics such as ketanserine; thromboxane synthetase inhibitors; neutral endopeptidase inhibitors (NEP inhibitors); angiotensin II antagonists; and diuretics such as hydrochlorothiazide, chlorothiazide, acetazolamide, amiloride, bumetanide, benzthiazide, ethacrynic acid, furosemide, indacrinone, metolazone, spironolactone, triamterene, chlorthalidone etc.; sympatholytics such as methyldopa, clonidine, guanabenz, reserpine; and other agents suitable for the treatment of high blood pressure, heart failure or vascular disorders associated with diabetes or renal disorders such as acute or chronic renal failure in humans and animals. Such combinations can be used separately or in products which comprise a plurality of components.

Further substances which can be used in combination with the compounds of the formulae (I) or (IA) are the compounds of classes (i) to (ix) on page 1 of WO 02/40007 (and the preferences and examples detailed further therein) and the substances mentioned on pages 20 and 21 of WO 03/027091.

The dosage may vary within wide limits and must of course be adapted to the individual circumstances in each individual case. In general, a daily dose appropriate for oral administration ought to be from about 3 mg to about 3 g, preferably about 10 mg to about 1 g, e.g. approximately 300 mg per adult person (70 kg), divided into preferably 1-3 single doses, which may be for example of equal size, although the stated upper limit may also be exceeded if this proves to be indicated, and children usually receive a reduced dose appropriate for their age and body weight.

### EXAMPLES

The following examples illustrate the present invention. All temperatures are stated in degrees Celsius and pressures in mbar. Unless mentioned otherwise, the reactions take place at RT. The abbreviation “Rf=xx(A)” means for example that the Rf xx was found in solvent system A. The ratio of amounts of solvents to one another is always indicated in proportions by volume. Chemical names for final products and intermediates were generated with the aid of the AutoNom 2000 (Automatic Nomenclature) program.

Thin-layer chromatography element systems:
A CH₂Cl₂/MeOH/NH₃ 25%=200:20:1
B CH₂Cl₂/MeOH/NH₃ 25%=200:20:0.5
C CH₂Cl₂/MeOH/NH₃ 25%=200:10:1
D CH₂Cl₂/MeOH/NH₃ 25%=90:10:1
E CH₂Cl₂/MeOH/NH₃ 25%=60:10:1
F CH₂Cl₂/MeOH/NH₃ 25%=200:30:1
G CH₂Cl₂/MeOH=9:1
H CH₂Cl₂/MeOH/NH₃ 25%=200:15:1
I CH₂Cl₂/MeOH/NH₃ 25%=100:10:1

HPLC gradients on Hypersil BDS C-18 (5 um); column: 4×125 mm
I 90% H₂O*/10% CH₃CN* to 0% H₂O*/100% CH₃CN* in 5 min+2.5 min (1.5 ml/min)
II 95% H₂O*/5% CH₃CN* to 0% H₂O*/100% CH₃CN* in 30 min+5 min (0.8 ml/min) * contains 0.1% trifluoroacetic acid

The following abbreviations are used:
AcOH acetic acid
n-BuLi n-butyllithium
t-BuOH tert-butanol
CH₂Cl₂ dichloromethane
CHCl₃ chloroform
CH₃CN acetonitrile
Cy cyclohexane
DCC dicyclohexylcarbodiimide
DIBAL diisobutylaluminium hydride
DME 1,2-dimethoxyethane
DMF N,N-dimethylformamide
EDC.HCl N-ethyl-N′-(3-dimethylaminopropyl)carbodiimide hydrochloride [25952-53-8]
Et₃N triethylamine
Et₂O diethylether
EtOAc ethyl acetate
EtOH ethanol
h hour(s)
HBr hydrobromic acid
HCl hydrochloric acid
H₂O water
K₂CO₃ potassium carbonate
LiCl lithium chloride
LiAlH₄ lithium aluminium hydride
MeI methyl iodide
MeOH methanol
MeONa sodium methylate
min minute(s)
m.p. melting point (temperature)
N₂ nitrogen
Na₂CO₃ sodium carbonate
NaH sodium hydride
NaHCO₃ sodium bicarbonate
NaOH sodium hydroxide
Na₂SO₄ sodium sulfate
NH₃ ammonia
NH₄Br ammonium bromide
NH₄Cl ammonium chloride
NH₄OH ammonium hydroxide
Pd₂(dba)₃ tris(dibenzylideneacetone)dipalladium [51364-51-3]
Pd(PPh₃)₄ tetrakis-triphenylphosphine palladium(0)
P(tert-Bu)₃ tri-tert-butylphosphine
Ra/Ni Raney-nickel
Rf ratio of distance which a substance travels to distance of the eluent front from the start point in thin layer chromatography
Rt retention time of a substance in HPLC (in minutes)
RT room temperature (23° C.)
TBME tert-butyl methyl ether
TFA trifluoroacetic acid
THF tetrahydrofuran

General Procedure J (N-Tos-Deprotection I)

To a stirred solution of 0.09 mmol “tosylamide” in 10 ml of MeOH are added 0.44 mmol sodiumdihydrogenphosphate and 0.90 mmol of sodium amalgam (10% Na) at RT. The reaction mixture is stirred for 2-18 h, diluted with water and extracted with EtOAc. The organic phases are combined, washed with brine and dried over Na₂SO₄. The solvent is concentrated under reduced pressure and the residue is purified by flash chromatography (SiO₂ 60 F) to afford the title compound.

### Example 1

### 6-{(3S,4S)-4-(2-Methoxy-ethoxy)-4-[4-(2-methoxy-ethoxymethyl)-phenyl]-piperidine-3-yloxymethyl}-4-(3-methoxy-propyl)-3,4-dihydro-2H-benzo[1,4]oxazine

To a solution of 0.33 mmol of (3S,4S)-4-(2-methoxy-ethoxy)-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester in 2 ml of CH₂Cl₂ is added at 0° C. 6.58 mmol of TFA and the reaction mixture is stirred at RT for 45 min (conversion checked by HPLC or TLC). The reaction mixture is poured into ice-cold saturated aqueous NaHCO₃ (20 ml) and extracted with CH₂Cl₂ (2×100 ml). The combined organic layers are dried over Na₂SO₄ and evaporated under reduced pressure. The title compound is obtained from the residue by flash chromatography (SiO₂ 60 F) as a yellow oil. Rf=0.27 (CH₂Cl₂/MeOH/NH₃ 25% 200:20:1); Rt=3.49 (gradient I).

The starting material(s) is(are) prepared as follows:

### a) (3S,4S)-4-(2-Methoxy-ethoxy)-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

1.45 mmol of NaH (60% dispersion in oil) are added to a solution of 0.97 mmol of (3S,4S)-4-hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester, 0.97 mmol tetrabutylammonium iodide and 1.45 mmol of 2-bromoethyl methyl ether in 17 ml of DMF. The reaction mixture is stirred at 0° C. for 1 h and at RT for 18 h. The mixture is then poured into 1M aqueous NaHCO₃ (100 ml) and extracted with TBME (2×150 ml). The combined organic layers are washed successively with H₂O (2×80 ml) and brine (1×80 ml), dried over Na₂SO₄ and evaporated under reduced pressure. The title compound is obtained from the residue by flash chromatography (SiO₂ 60 F) as a yellow oil. Rf=0.25 (EtOAc/heptane 1:1); Rt=5.05 (gradient I).

### b) (3S,4S)-4-Hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

A solution of 1.27 mmol of (3S,4S)-4-hydroxy-4-[4-(2-methoxy-ethoxymethyl)-phenyl]-3-[4-(3-methoxypropyl)-3-oxo-3,4-dihydro-2H-benzo[1,4]oxazin-6-ylmethoxy]-piperidine-1-carboxylic acid tert-butyl ester in 22 ml of THF is mixed with 4.0 mmol of borane-THF complex (1 M in THF) and stirred at RT for 3 days (conversion checked by HPLC or TLC). After addition of 15 ml of MeOH, the reaction mixture is evaporated under reduced pressure. The title compound is obtained from the residue by flash chromatography (SiO₂ 60 F) as a yellow oil. Rf (EtOAc/heptane 1:1)=0.31, Rt=4.80 (gradient I).

### c) (3S,4S)-4-Hydroxy-4-[4-(2-methoxy-ethoxymethyl)-phenyl]-3-[4-(3-methoxypropyl)-3-oxo-3,4-dihydro-2H-benzo[1,4]oxazin-6-ylmethoxy]-piperidine-1-carboxylic acid tert-butyl ester

7.4 mmol of NaH (60% dispersion in oil) are added to a solution of 6.7 mmol of (3S,4S)-3,4-dihydroxy-4-[4-(2-methoxy-ethoxymethyl)-phenyl]-piperidine-1-carboxylic acid tert-butyl ester, 7.4 mmol tetrabutylammonium iodide and 7.1 mmol of 6-bromomethyl-4-(3-methoxy-propyl)-4H-benzo[1,4]oxazin-3-one in 25 ml of DMF while the reaction mixture is stirred at 0° C. for 1 h and at RT for 18 h. The mixture is poured into 1M aqueous NaHCO₃ (100 ml) and extracted with CH₂Cl₂ (2×150 ml). The combined organic layers are washed successively with H₂O (2×80 ml) and brine (1×80 ml), dried over Na₂SO₄ and evaporated under reduced pressure. The title compound is obtained from the residue by flash chromatography (SiO₂ 60 F) as a yellow oil. Rf (EtOAc/heptane 2:1)=0.32; Rt=4.48 (gradient I).

### d) (3S,4S)-3,4-Dihydroxy-4-[4-(2-methoxy-ethoxymethyl)-phenyl]-piperidine-1-carboxylic acid tert-butyl ester

To a stirred solution of (38.3 g) of AD-mix-α [ALDRICH, 39,275-8, lot 01614BE/277] in 80 ml of t-BuOH and 80 ml of H₂O is added 22.4 mmol of methanesulfonamide. The reaction mixture is cooled to 0° C. followed by the addition of 22.4 mmol of 4-[4-(2-methoxy-ethoxymethyl)-phenyl]-3,6-dihydro-2H-pyridine-1-carboxylic acid tert-butyl ester in 35 ml of t-BuOH and 35 ml of H₂O. The reaction mixture is stirred at 0° C. for 30 min and allowed to stir at RT for 3 days. To the reaction mixture is added 33 g of Na₂SO₃ followed by stirring for 1 h. CH₂Cl₂ (250 ml) is added, the layers are separated and the aqueous layer is extracted again with CH₂Cl₂ (4×150 ml). The combined organic layers are washed with 2N aqueous KOH (200 ml), dried over Na₂SO₄ and evaporated under reduced pressure. The title compound is obtained from the residue by flash chromatography (SiO₂ 60 F) as a yellow oil. Rf=0.06 (EtOAc/heptane 1:2); Rt=3.52 (gradient I).

### e) 4-[4-(2-Methoxy-ethoxymethyl)-phenyl]-3,6-dihydro-2H-pyridine-1-carboxylic acid tert-butyl ester

A three neck flask is charged with 22.2 mmol of 4-trifluoromethanesulfonyloxy-3,6-dihydro-2H-pyridine-1-carboxylic acid tert-butyl ester [138647-49-1], 30.2 mmol of 4-(2-methoxy-ethoxymethyl)-phenylboronic acid, 66.7 mmol of LiCl, 105 ml of 2N aqueous Na₂CO₃, 220 ml of DME and 1.1 mmol of Pd(PPh₃)₄. The reaction is heated to reflux for 3 h followed by cooling to RT and concentration under reduced pressure. The resulting residue is partitioned between CH₂Cl₂ (500 ml), 2N aqueous Na₂CO₃ (400 ml) and concentrated NH₄OH (25 ml). The layers are separated and the aqueous layer is extracted again with CH₂Cl₂ (3×500 ml). The combined organic layers are dried over Na₂SO₄ and evaporated under reduced pressure. The title compound is obtained from the residue by flash chromatography (SiO₂ 60 F) as a yellow oil. Rf=0.50 (EtOAc/heptane 1:1); Rt=4.81 (gradient I).

### f) 4-(2-Methoxy-ethoxymethyl)-phenylboronic acid

A solution of 38.8 mmol of n-BuLi (1.6 M in hexanes) is added dropwise to a stirred solution of 32.3 mmol of 1-bromo-4-(2-methoxy-ethoxymethyl)-benzene [166959-29-1] in 50 ml of THF at −78° C. The reaction mixture is stirred for 30 min at −78° C. and 64.6 mmol of triisopropyl borate are added rapidly. The mixture is stirred for 30 min at −78° C. and at RT for 1 h. The reaction mixture is partitioned between 2N aqueous HCl (40 ml) and EtOAc (300 ml). The organic layer is washed with brine (2×50 ml), dried over Na₂SO₄ and evaporated under reduced pressure to afford the title compound as a yellow oil. Rt=2.52.

According to the process described in example 1, the following compounds are prepared in an analogous manner:

### 6 6-{(3S,4S)-4-Cyclopropylmethoxy-4-[4-(2-methoxy-ethoxymethyl)-phenyl]-piperidin-3-yloxymethyl}-4-(3-methoxy-propyl)-3,4-dihydro-2H-benzo[1,4]oxazine

using bromomethylcyclopropane instead of 2-bromoethyl methyl ether in step a. Yellowish solid; Rf=0.25 (CH₂Cl₂/MeOH/NH₃ 25% 200:15:1); Rt=4.05 (gradient I).

### 7 6-[(3S,4S)-4-[4-(2-Methoxy-ethoxymethyl)-phenyl]-4-(3-methoxy-propoxy)-piperidin-3-yloxymethyl]-4-(3-methoxy-propyl)-3,4-dihydro-2H-benzo[1,4]oxazine

using 1-bromo-3-methoxypropane instead of 2-bromoethyl methyl ether in step a. Yellowish solid; Rf=0.43 (CH₂Cl₂/MeOH/NH₃ 25% 200:15:1); Rt=377 (gradient I)

### Example 2

### (R)-1-Methoxy-3-{(3S,4S)-4-[4-(2-methoxy-ethoxymethyl)-phenyl]-3-[4-(3-methoxy-propyl)-3,4-dihydro-2H-benzo[1,4]oxazin-6-ylmethoxy]-piperidin-4-yloxy}-propan-2-ol

According to example 1, (3S,4S)-4-((R)-2-hydroxy-3-methoxy-propoxy)-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellow oil. Rf=0.22 (CH₂Cl₂/MeOH/NH₃ 25% 200:20:1); Rt=3.33 (gradient I).

The starting material(s) is(are) prepared as follows:

### a) (3S,4S)-4-((R)-2-Hydroxy-3-methoxy-propoxy)-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

0.117 ml of a solution of MeONa in MeOH (30%, 0.904 mmol) is added to a solution of 0.822 mmol of (3S,4S)-4-[4-(2-methoxy-ethoxymethyl)-phenyl]-3-[4-(3-methoxy-propyl)-3,4-dihydro-2H-benzo[1,4]oxazin-6-ylmethoxy]-4-((R)-1-oxiranylmethoxy)-piperidine-1-carboxylic acid tert-butyl ester in 7 ml of MeOH. The reaction mixture is stirred at RT for 15 h then at 55° C. for 5 h 30 min. The mixture is evaporated under reduced pressure. The residue is partitioned between TBME (100 ml) and water (50 ml). The organic phase is separated and washed with brine (30 ml). The combined aqueous layers are extracted with TBME (50 ml). The combined organic layers are dried over Na₂SO₄ and evaporated under reduced pressure. The title compound is obtained from the residue by flash chromatography (SiO₂ 60 F) as a yellow oil. Rf=0.10 (EtOAc); Rt=4.76 (gradient I).

### b) (3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-4-((R)-1-oxiranylmethoxy)-piperidine-1-carboxylic acid tert-butyl ester

2.563 mmol of NaH (60% dispersion in oil) are added to a solution of 1.165 mmol of (3S,4S)-4-hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 1b) in 10 ml of THF. The mixture is heated to 55° C. and a solution of 2.33 mmol of (2R)-(−)-glycidyltosylate [113826-06-5] in 5 ml of THF is added drop-wise. The reaction mixture is stirred at 55° C. for 90 min, then 2.563 mmol of NaH (60% dispersion in oil) and 2.33 mmol of (2R)-(−)-glycidyltosylate are added. After 90 min of stirring at 55° C. the mixture is cooled to RT, diluted with TBME (100 ml) and washed with aqueous saturated NaHCO₃ (60 ml). The aqueous phase is separated and extracted with TBME (60 ml). The combined organic layers are washed successively with H₂O (1×50 ml) and brine (1×30 ml), dried over Na₂SO₄ and evaporated under reduced pressure. The title compound is obtained from the residue by flash chromatography (SiO₂ 60 F) as a yellow oil. Rf (EtOAc/heptane 3:1)=0.28; Rt=5.21 (gradient I).

According to the process described in example 2, the following compound is prepared in an analogous manner:

### 3 (S)-1-Methoxy-3-{(3S,4S)-4-[4-(2-methoxy-ethoxymethyl)-phenyl]-3-[4-(3-methoxy-propyl)-3,4-dihydro-2H-benzo[1,4]oxazin-6-ylmethoxy]-piperidin-4-yloxy}-propan-2-ol

using (2S)-(+)-glycidyltosylate [70987-78-9] instead of (2R)-(−)-glycidyltosylate in step b. Yellow oil; Rf=0.22 (CH₂Cl₂/MeOH/NH₃ 25% 200:20:1); Rt=3.31 (gradient I).

### Example 4

### 6-[(3S,4S)-4-[4-(2-Methoxy-ethoxymethyl)-phenyl]-4-(tetrahydro-pyran-4-ylmethoxy)-piperidin-3-yloxymethyl]-4-(3-methoxy-propyl)-3,4-dihydro-2H-benzo[1,4]oxazine

According to example 1, (3S,4S)-4-[4-(2-methoxy-ethoxymethyl)-phenyl]-3-[4-(3-methoxy-propyl)-3,4-dihydro-2H-benzo[1,4]oxazin-6-ylmethoxy]-4-(tetrahydro-pyran-4-ylmethoxy)-piperidine-1-carboxylic acid tert-butyl ester is used to afford the title compound as a yellowish solid. Rf=0.41 (CH₂Cl₂/MeOH/NH₃ 25% 200:15:1); Rt=3.79 (gradient I).

The starting material(s) is(are) prepared as follows:

### a) (3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-4-(tetrahydro-pyran-4-ylmethoxy)-piperidine-1-carboxylic acid tert-butyl ester

0.998 mmol of NaH (60% dispersion in oil) are added to a solution of 0.832 mmol of (3S,4S)-4-hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 1b) in 2 ml of DMF. The mixture is stirred at 40° C. for 25 min, then a solution of 1.248 mmol of toluene-4-sulfonic acid tetrahydro-pyran-4-ylmethyl ester [101691-65-0] in 1 ml of DMF is added. The reaction mixture is stirred at 40° C. for 24 h then 0.2 mmol of toluene-4-sulfonic acid tetrahydro-pyran-4-ylmethyl ester and 0.45 mmol of NaH (60% dispersion in oil) are added. The reaction mixture is stirred at 40° C. for 60 h an is then cooled to RT. The mixture is diluted with TBME (100 ml), washed successively with aqueous saturated NaHCO₃ (20 ml) and brine (20 ml), dried over Na₂SO₄ and evaporated under reduced pressure. The title compound is obtained from the residue by flash chromatography (SiO₂ 60 F) as a colorless solid. Rf (EtOAc/heptane 1:1)=0.21; Rt=5.57 (gradient I).

According to the process described in example 4, the following compound is prepared in an analogous manner:

### 5 6-{(3S,4S)-4-Cyclopentylmethoxy-4-[4-(2-methoxy-ethoxymethyl)-phenyl]-piperidin-3-yloxymethyl}-4-(3-methoxy-propyl)-3,4-dihydro-2H-benzo[1,4]oxazine

using toluene-4-sulfonic acid cyclopentylmethyl ester [21856-53-1] instead of toluene-4-sulfonic acid tetrahydro-pyran-4-ylmethyl ester in step a. Yelloish solid; Rf=0.12 (CH₂Cl₂/MeOH/NH₃ 25% 200:10:1); Rt=4.64 (gradient I)

### Example 8

### (R)-1-{(3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-piperidin-4-yloxy}-propan-2-ol

According to example 1, (3S,4S)-4-((R)-2-hydroxy-propoxy)-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellow oil. Rf=0.17 (CH₂Cl₂/MeOH/NH₃ 25% 200:20:1); Rt=3.38 (gradient I)

The starting material(s) is(are) prepared as follows:

### a) (3S,4S)-4-((R)-2-Hydroxy-propoxy)-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

2.802 mmol of NaBH₄ are added to a solution of 0.934 mmol of (3S,4S)-4-[4-(2-methoxy-ethoxymethyl)-phenyl]-3-[4-(3-methoxy-propyl)-3,4-dihydro-2H-benzo[1,4]oxazin-6-ylmethoxy]-4-((R)-1-oxiranylmethoxy)-piperidine-1-carboxylic acid tert-butyl ester (example 2b) in 12.5 ml of EtOH and 1 ml of THF. The reaction mixture is stirred for 16 h at 45° C. then it's cooled to RT and partitioned between TBME (100 ml) and aqueous saturated NH₄Cl (70 ml). The aqueous phase is separated and extracted with TBME (50 ml). The combined organic layers are washed successively with water (50 ml) and brine (30 ml), dried over Na₂SO₄ and evaporated under reduced pressure. The title compound is obtained from the residue by flash chromatography (SiO₂ 60 F) as a yellow oil. Rf (EtOAc/heptane 2:1)=0.11; Rt=4.83 (gradient I).

According to the process described in example 8, the following compound is prepared in an analogous manner:

### 9 (S)-1-{(3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-piperidin-4-yloxy}-propan-2-ol

using (2S)-(+)-glycidyltosylate [70987-78-9] instead of (2R)-(−)-glycidyltosylate in the step analogous to step b (example 2). Yellow oil; Rf=0.17 (CH₂Cl₂/MeOH/NH₃ 25% 200:20:1); Rt=3.29 (gradient I).

### 17 (R)-1-Methoxy-3-{(3S,4S)-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidin-4-yloxy}-propan-2-ol

using (3S,4S)-4-hydroxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 10 b) instead of (3S,4S)-4-hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester and S-(+)-glycidyl methyl ether [64491-68-5] instead of (2R)-(−)-glycidyltosylate in the step analogous to step b (example 2).

### 18 (S)-1-Methoxy-3-{(3S,4S)-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidin-4-yloxy}-propan-2-ol

using (3S,4S)-4-hydroxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 10 b) instead of (3S,4S)-4-hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester and R-(−)-glycidyl methyl ether [64491-70-9] instead of (2R)-(−)-glycidyltosylate in the step analogous to step b (example 2).

### 19 (R)-1-{(3S,4S)-4-[4-((S)-3-Methoxy-2-methyl-propoxymethyl)-phenyl]-3-[4-(3-methoxy-propyl)-3,4-dihydro-2H-benzo[1,4]oxazin-6-ylmethoxy]-piperidin-4-yloxy}-propan-2-ol

using (3S,4S)-4-hydroxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 10 b) instead of (3S,4S)-4-hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester in the step analogous to step b (example 2).

### 20 (S)-1-{(3S,4S)-4-[4-((S)-3-Methoxy-2-methyl-propoxymethyl)-phenyl]-3-[4-(3-methoxy-propyl)-3,4-dihydro-2H-benzo[1,4]oxazin-6-ylmethoxy]-piperidin-4-yloxy}-propan-2-ol

starting from (3S,4S)-4-hydroxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 10 b) instead of (3S,4S)-4-hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester and (2S)-(+)-glycidyltosylate [70987-78-9] instead of (2R)-(−)-glycidyltosylate in the step analogous to step b (example 2).

### Example 10

### 6-[(3S,4S)-4-[4-((S)-3-Methoxy-2-methyl-propoxymethyl)-phenyl]-4-(3-methoxy-propoxy)-piperidin-3-yloxymethyl]-4-(3-methoxy-propyl)-3,4-dihydro-2H-benzo[1,4]oxazine

According to example 1, (3S,4S)-4-[4-((S)-3-methoxy-2-methyl-propoxymethyl)-phenyl]-4-(3-methoxy-propoxy)-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellow oil. Rf=0.24 (CH₂Cl₂/MeOH/NH₃ 25% 200:20:1); Rt=4.35 (gradient I).

The starting material(s) is(are) prepared as follows:

### a) (3S,4S)-4-[4-((S)-3-Methoxy-2-methyl-propoxymethyl)-phenyl]-4-(3-methoxy-propoxy)-3-[4-(3-methoxy-propyl)-3,4-dihydro-2H-benzo[1,4]oxazin-6-ylmethoxy]-piperidine-1-carboxylic acid tert-butyl ester

According to example 1a, (3S,4S)-4-hydroxy-4-[4-((S)-3-methoxy-2-methyl-propoxy-methyl)-phenyl]-3-[4-(3-methoxy-propyl)-3,4-dihydro-2H-benzo[1,4]oxazin-6-yl-methoxy]-piperidine-1-carboxylic acid tert-butyl ester and 1-bromo-3-methoxypropane [36865-41-5] are used to afford the title compound as a yellow oil. Rf (EtOAc/heptane 1:1)=0.21; Rt=6.03 (gradient I).

### b) (3S,4S)-4-Hydroxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

According to example 1b, (3S,4S)-4-hydroxy-4-[4-((S)-3-methoxy-2-methyl-propoxy-methyl)-phenyl]-3-[4-(3-methoxy-propyl)-3-oxo-3,4-dihydro-2H-benzo[1,4]oxazin-6-ylmethoxy]-piperidine-1-carboxylic acid tert-butyl ester is used to afford the title compound as a yellow oil. Rf (EtOAc/heptane 1:1)=0.28; Rt=5.56 (gradient I).

### c) (3S,4S)-4-Hydroxy-4-[4-((S)-3-methoxy-2-methyl-propoxymethyl)-phenyl]-3-[4-(3-methoxy-propyl)-3-oxo-3,4-dihydro-2H-benzo[1,4]oxazin-6-ylmethoxy]-piperidine-1-carboxylic acid tert-butyl ester

According to example 1c, (3S,4S)-3,4-dihydroxy-4-[4-((S)-3-methoxy-2-methyl-propoxymethyl)-phenyl]-piperidine-1-carboxylic acid tert-butyl ester is used to afford the title compound as a yellow oil. Rf (EtOAc/heptane 1:1)=0.12; Rt=5.19 (gradient I).

### d) (3S,4S)-3,4-Dihydroxy-4-[4-((S)-3-methoxy-2-methyl-propoxymethyl)-phenyl]-piperidine-1-carboxylic acid tert-butyl ester

According to example 1d, 4-[4-((S)-3-methoxy-2-methyl-propoxymethyl)-phenyl]-3,6-dihydro-2H-pyridine-1-carboxylic acid tert-butyl ester is used to afford the title compound as a yellow oil. Rf (EtOAc/heptane 1:2)=0.10; Rt=4.25 (gradient I).

### e) 4-[4-((S)-3-Methoxy-2-methyl-propoxymethyl)-phenyl]-3,6-dihydro-2H-pyridine-1-carboxylic acid tert-butyl ester

According to example 1e, 4-((R)-3-methoxy-2-methyl-propan-1-oxymethyl)-phenylboronic acid and 4-trifluoromethanesulfonyloxy-3,6-dihydro-2H-pyridine-1-carboxylic acid tert-butyl ester [138647-49-1] are used to afford the title compound as a yellow oil. Rf (EtOAc/heptane 1:4)=0.25; Rt=5.69 (gradient I).

### f) 4-((R)-3-Methoxy-2-methyl-propan-1-oxymethyl)-phenylboronic acid

According to example 1f, 1-bromo-4-((S)-3-methoxy-2-methyl-propoxymethyl)-benzene is used to afford the title compound as a yellow oil. Rt=3.36 (gradient I).

g) 1-Bromo-4-((S)-3-methoxy-2-methyl-propoxymethyl)-benzene

A solution of 501.9 mmol of (R)-3-methoxy-2-methyl-propan-1-ol [911855-78-2] in 100 ml of DMF is added for 30 min to an ice-cooled suspension of 602.2 mmol of NaH (60% dispersion in oil) in 250 ml of DMF. The suspension is stirred at 0° C. for 30 min then a solution of 401.5 mmol of 1-bromo-4-chloromethyl-benzene in 100 ml of THF is added for 30 min. The reaction mixture is stirred at RT for 4 h, diluted with TBME (0.75 l) and washed with aqueous saturated NaHCO₃ (750 ml). The aqueous phase is extracted with TBME (3×1 l). The combined organic layers are washed successively with water (350 ml) and brine (350 ml), dried over Na₂SO₄ and evaporated under reduced pressure. The title compound is obtained from the residue by flash chromatography (SiO₂ 60 F) as a yellowish oil. Rf (EtOAc/heptane 1:6)=0.43; Rt=5.28 (gradient I).

### Example 11

### 2-{(3S,4S)-4-[4-((S)-3-Methoxy-2-methyl-propoxymethyl)-phenyl]-3-[4-(3-methoxy-propyl)-3,4-dihydro-2H-benzo[1,4]oxazin-6-ylmethoxy]-piperidin-4-yloxy}-N,N-dimethyl-acetamide

According to example 1, (3S,4S)-4-dimethylcarbamoylmethoxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellow oil. Rf=0.22 (CH₂Cl₂/MeOH/NH₃ 25% 100:10:1); Rt=3.75 (gradient I).

The starting material(s) is(are) prepared as follows:

### a) (3S,4S)-4-Dimethylcarbamoylmethoxy-4-[4-((S)-3-methoxy-2-methyl-propoxy-methyl)-phenyl]-3-[4-(3-methoxy-propyl)-3,4-dihydro-2H-benzo[1,4]oxazin-6-ylmethoxy]-piperidine-1-carboxylic acid tert-butyl ester

A solution of 0.408 mmol of (3S,4S)-4-ethoxycarbonylmethoxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester in 11 ml of dimethylamine in EtOH (33%, 81 mmol) is stirred for 60 h at 50° C. in an air-tight Supelco vial (22 ml). The reaction mixture is then evaporated to dryness. The title compound is obtained from the residue by flash chromatography (SiO₂ 60 F) as a yellow oil. Rf (EtOAc/MeOH 20:1)=0.38, Rt=5.18 (gradient I).

### b) (3S,4S)-4-Ethoxycarbonylmethoxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

3.02 mmol of potassium hydride (free from oil, freshly prepared from a 30% dispersion in oil washed with pentane and dried in vacuo) is added to a solution of 1.51 mmol of (3S,4S)-4-hydroxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 10b) in 13 ml of THF. The suspension is stirred for 25 min at RT then a solution of 4.53 mmol of ethyl bromoacetate in 2 ml of THF is added. The reaction mixture is stirred for 90 min at RT, then 3.02 mmol of potassium hydride and 4.53 mmol of ethyl bromoacetate are further added. The reaction mixture is stirred at RT for 18 h, diluted with TBME (200 ml) and washed with aqueous saturated NaHCO₃ (40 ml). The aqueous phase is extracted with TBME (150 ml). The combined organic layers are washed successively with water (30 ml) and brine (20 ml), dried over Na₂SO₄ and evaporated under reduced pressure. To afforded the crude title compound as a yellow oil. Rt=5.96 (gradient I).

According to the process described in example 11, the following compound is prepared in an analogous manner:

### 15 2-{(3S,4S)-4-[4-((S)-3-Methoxy-2-methyl-propoxymethyl)-phenyl]-3-[4-(3-methoxy-propyl)-3,4-dihydro-2H-benzo[1,4]oxazin-6-ylmethoxy]-piperidin-4-yloxy}-N-methyl-acetamide

using a solution of methylamine (33% in EtOH) instead of dimethylamine in step a (stirring at 70° C.). Yellow oil; Rf=0.08 (CH₂Cl₂/MeOH/NH₃ 25% 200:20:1); Rt=3.62 (gradient I).

### 16 2-{(3S,4S)-4-[4-((R)-2-Ethoxy-propoxymethyl)-phenyl]-3-[4-(3-methoxy-propyl)-3,4-dihydro-2H-benzo[1,4]oxazin-6-ylmethoxy]-piperidin-4-yloxy}-N-methyl-acetamide

using (3S,4S)-4-[4-((R)-2-ethoxy-propoxymethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester instead of (3S,4S)-4-hydroxy-4-[4-((S)-3-methoxy-2-methyl-propoxy-methyl)-phenyl]-3-[4-(3-methoxy-propyl)-3,4-dihydro-2H-benzo[1,4]oxazin-6-ylmethoxy]-piperidine-1-carboxylic acid tert-butyl ester in step b and a solution of methylamine (33% in EtOH) instead of dimethylamine in step a.

The starting material(s) is(are) prepared as follows:

### a) (3S,4S)-4-[4-((R)-2-Ethoxy-propoxymethyl)-phenyl]-4-hydroxy-3-[4-(3-methoxy-propyl)-3,4-dihydro-2H-benzo[1,4]oxazin-6-ylmethoxy]-piperidine-1-carboxylic acid tert-butyl ester

According to example 1 (steps b-e) and starting from [4-((R)-2-ethoxy-propoxy-methyl)-phenyl]-dimethyl-boronic acid the title compound is obtained as a yellowish solid. Rf (EtOAc/heptane 1:1)=0.21; Rt=5.48 (gradient I).

### b) [4-((R)-2-Ethoxy-propoxymethyl)-phenyl]-dimethyl-boronic acid

According to example 10g, 1-bromo-4-((R)-2-ethoxy-propoxymethyl)-benzene is used to afford the title compound as a yellow-greenish oil. Rt=3.16 (gradient I).

### c) 1-Bromo-4-((R)-2-ethoxy-propoxymethyl)-benzene

101.65 mmol of NaH (55% dispersion in oil) are added to a solution of 61.60 mmol of (R)-1-(4-bromo-benzyloxy)-propan-2-ol in 115 ml of DMF. The reaction mixture is stirred for 1 h at RT then 110.89 mmol of ethyliodide are added over 5 min. The reaction mixture is stirred at RT for 18 h, then poured into saturated aqueous NH₄Cl (200 ml) and extracted with TBME (2×250 ml). The combined organic layers are washed successively with H₂O (2×100 ml) and brine (1×100 ml), dried over Na₂SO₄ and evaporated under reduced pressure. The title compound is obtained from the residue by flash chromatography (SiO₂ 60 F) as a yellow oil. Rf (EtOAc/heptane 1:2)=0.63; Rt=4.85 (gradient I).

### d) (R)-1-(4-Bromo-benzyloxy)-propan-2-ol

A solution containing 66.23 mmol of (R)-2-(4-bromo-benzyloxymethyl)-oxirane and 397.36 mmol of NaBH₄ in 165 ml of ethanol and 16.5 ml of THF is stirred at 55° C. for 3 h, cooled to RT then poured into 700 ml of cold 1N NH₄Cl. The mixture is extracted with TBME (2×700 ml). The combined organic layers are washed with brine (700 ml), dried over Na₂SO₄, filtered and evaporated under reduced pressure. The title compound is obtained oil from the residue by flash chromatography (SiO₂ 60 F) as a colourless. Rf (EtOAc/heptane 1:1)=0.50; Rt=3.77 (gradient I).

### Example 12

### {(3S,4S)-4-[4-((S)-3-Methoxy-2-methyl-propoxymethyl)-phenyl]-3-[4-(3-methoxy-propyl)-3,4-dihydro-2H-benzo[1,4]oxazin-6-ylmethoxy]-piperidin-4-yloxy}-acetonitrile

According to example 1, (3S,4S)-4-cyanomethoxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellow oil. Rf=0.25 (CH₂Cl₂/MeOH/NH₃ 25% 200:20:1); Rt=4.11 (gradient I).

The starting material(s) is(are) prepared as follows:

### a) (3S,4S)-4-Cyanomethoxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

0.679 mmol of NaH (60% dispersion in oil) is added to a solution of 0.453 mmol of (3S,4S)-4-hydroxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 10b) in 3 ml of CH₃CN. The milky mixture is stirred for 1 h at RT then the mixture is cooled to 0° C. 0.906 mmol of bromoacetonitrile are added and the reaction mixture is stirred at RT. After 2 h, NaH then bromoacetonitrile (same quantities as above) are added. This procedure is repeated once. The reaction mixture is diluted with TBME (100 ml) and washed with aqueous saturated NaHCO₃ (30 ml). The aqueous phase is extracted with TBME (100 ml). The combined organic layers are washed successively with water (20 ml) and brine (15 ml), dried over Na₂SO₄ and evaporated under reduced pressure. The title compound is obtained from the residue by flash chromatography (SiO₂ 60 F) as a colourless oil. Rf (EtOAc/heptane 1:1)=0.23; Rt=5.82 (gradient I).

### Example 13

### 1-{(3S,4S)-4-[4-((S)-3-Methoxy-2-methyl-propoxymethyl)-phenyl]-3-[4-(3-methoxy-propyl)-3,4-dihydro-2H-benzo[1,4]oxazin-6-ylmethoxy]-piperidin-4-yloxy}-2-methyl-propan-2-ol

According to example 1, (3S,4S)-4-(2-hydroxy-2-methyl-propoxy)-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester is used to afford the title compound as a yellow oil. Rf=0.24 (CH₂Cl₂/MeOH/NH₃ 25% 200:20:1); Rt=3.92 (gradient I).

The starting material(s) is(are) prepared as follows:

### a) (3S,4S)-4-(2-Hydroxy-2-methyl-propoxy)-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester

0.981 mmol of methylmagnesium bromide (3N in Et₂O, 0.328 ml) are added to an ice-cooled solution of 0.218 mmol of (3S,4S)-4-ethoxycarbonylmethoxy-4-[4-((S)-3-methoxy-2-methyl-propoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 11b) in 20 ml of THF. The reaction mixture is stirred for 10 min at 0° C. then for 1 h at RT. The mixture is diluted with CH₂Cl₂ (50 ml) and washed with aqueous saturated NaHCO₃ (20 ml). The aqueous phase is extracted with CH₂Cl₂ (2×50 ml). The combined organic layers are washed with brine (15 ml), dried over Na₂SO₄ and evaporated under reduced pressure. The title compound is obtained from the residue by flash chromatography (SiO₂ 60 F) as a colourless oil. Rf (EtOAc/heptane 1:1)=0.08; Rt=5.60 (gradient I).

### Example 14

### N-(2-{(3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-piperidin-4-yloxy}-ethyl)-acetamide

According to general procedure J, N-{2-[(3S,4S)-4-[4-(2-methoxy-ethoxymethyl)-phenyl]-3-[4-(3-methoxy-propyl)-3,4-dihydro-2H-benzo[1,4]oxazin-6-ylmethoxy]-1-(toluene-4-sulfonyl)-piperidin-4-yloxy]-ethyl}-acetamide is used to obtain the title compound as a yellow oil. Rf=0.10 (CH₂Cl₂/MeOH/NH₃ 25% 100:10:1); Rt=3.16 (gradient I).

The starting material(s) is(are) prepared as follows:

### a) N-{2-[(3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-1-(toluene-4-sulfonyl)-piperidin-4-yloxy]-ethyl}-acetamide

0.074 mmol of acetyl chloride are added to a solution of 0.067 mmol of 2-[(3S,4S)-4-[4-(2-methoxy-ethoxymethyl)-phenyl]-3-[4-(3-methoxy-propyl)-3,4-dihydro-2H-benzo[1,4]oxazin-6-ylmethoxy]-1-(toluene-4-sulfonyl)-piperidin-4-yloxy]-ethylamine and 0.08 mmol of triethylamine in 2 ml of CH₂Cl₂. The reaction mixture is stirred at RT for 40 min, then 6 ml of aqueous saturated NaHCO₃ and 3 ml of water are added. The mixture is extracted with CH₂Cl₂ (2×40 ml). The combined organic phases are dried over Na₂SO₄ and evaporated under reduced pressure. The crude title compound is obtained from the residue as a yellow oil. Rt=4.48 (gradient I).

### b) 2-[(3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-1-(toluene-4-sulfonyl)-piperidin-4-yloxy]-ethylamine

A solution of 0.819 mmol of [(3S,4S)-4-[4-(2-methoxy-ethoxymethyl)-phenyl]-3-[4-(3-methoxy-propyl)-3,4-dihydro-2H-benzo[1,4]oxazin-6-ylmethoxy]-1-(toluene-4-sulfonyl)-piperidin-4-yloxy]-acetonitrile in 5 ml of diethylether and 2 ml of THF is added dropwise to a solution of 0.901 mmol of LiAlH₄ (0.902 ml of a commercial solution, 1N in THF) in 6 ml of Et₂O. The reaction mixture is stirred for 30 min at RT, then successively 1 ml of 4N aqueous NaOH and 20 ml of aqueous saturated NaHCO₃ are carefully added. The mixture is extracted with EtOAc (3×50 ml). The combined organic layers are dried over Na₂SO₄ and evaporated under reduced pressure. The title compound is obtained from the residue by flash chromatography (SiO₂ 60 F) as a reddish oil. Rf (CH₂Cl₂/MeOH/NH₃ 25%=100:10:1)=0.24; Rt=4.20 (gradient I).

### c) [(3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-1-(toluene-4-sulfonyl)-piperidin-4-yloxy]-acetonitrile

1.918 mmol of NaH (60% dispersion in oil) are added to a solution of 0.959 mmol of (3S,4S)-4-[4-(2-methoxy-ethoxymethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol in 6 ml of THF and 1 ml of CH₃CN. The suspension is stirred for 40 min at RT then 2.877 mmol of bromoacetonitrile are added slowly. The mixture is stirred for 2 h at RT then 1.918 mmol of NaH (60% dispersion in oil) and, 40 min later, 2.977 mmol of bromo-acetonitrile are added to complete the reaction. After 15 h of stirring at RT, 10 ml of aqueous saturated NaHCO₃ then 30 ml of water are carefully added. The mixture is extracted with TBME (3×80 ml). The combined organic layers are washed with brine (30 ml), dried over Na₂SO₄ and evaporated under reduced pressure. The title compound is obtained from the residue by flash chromatography (SiO₂ 60 F) as a reddish oil. Rf (EtOAc/heptane 1:1)=0.15; Rt=5.20 (gradient I).

### d) (3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-1-(toluene-4-sulfonyl)-piperidin-4-ol

A solution of 4.794 mmol of p-toluenesulfonyl chloride in 10 ml of EtOAc is added dropwise to an ice-cold solution of 4.358 mmol of (3S,4S)-4-[4-(2-methoxyethoxy-methyl)-phenyl]-3-[4-(3-methoxy-propyl)-3,4-dihydro-2H-benzo[1,4]oxazin-6-ylmethoxy]-piperidin-4-ol in 15 ml of EtOAc and 25 ml of saturated aqueous NaHCO₃. After 1 h 30 min of stirring at 0° C., 100 ml of EtOAc and 50 ml of water are added. The organic phase is separated. The aqueous phase is extracted with EtOAc (50 ml). The combined organic layers are washed with water (50 ml) then brine (30 ml), dried over Na₂SO₄ and evaporated under reduced pressure. The title compound is obtained from the residue by flash chromatography (SiO₂ 60 F) as a yellow oil. Rf (EtOAc/heptane 2:1)=0.28; Rt=4.97 (gradient I).

### e) (3S,4S)-4-[4-(2-Methoxy-ethoxymethyl)-phenyl]-3-[4-(3-methoxy-propyl)-3,4-dihydro-2H-benzo[1,4]oxazin-6-ylmethoxy]-piperidin-4-ol

To an ice-cold solution of 5.826 mmol of (3S,4S)-4-hydroxy-4-[4-(2-methoxy-ethoxymethyl)-phenyl]-3-[4-(3-methoxy-propyl)-3,4-dihydro-2H-benzo[1,4]oxazin-6-ylmethoxy]-piperidine-1-carboxylic acid tert-butyl ester (example 1b) in 50 ml of CH₂Cl₂ are added 58.26 mmol of TFA and the reaction mixture is stirred from 0° C. to RT for 15 h. The reaction mixture is diluted with CH₂Cl₂ (100 ml) and washed with saturated aqueous NaHCO₃ (50 ml). The organic phase is separated and the aqueous phase is extracted with CH₂Cl₂ (50 ml). The combined organic layers are washed with water (50 ml) then brine (30 ml), dried over Na₂SO₄ and evaporated under reduced pressure. The crude title compound is obtained as a yellow oil. Rt=4.38 (gradient I).

1. A compound of the general formula 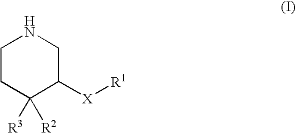
in which
R¹ is aryl or heterocyclyl, each of which is substituted by 1-4 radicals independently selected from the group consisting of
acyl-C₁₋₈-alkoxy-C₁₋₈-alkoxy,
acyl-C₁₋₈-alkoxy-C₁₋₈-alkyl,
(N-acyl)-C₁₋₈-alkoxy-C₁₋₈-alkylamino,
C₁₋₈-alkanoyl,
C₁₋₈-alkoxy,
C₁₋₈-alkoxy-C₁₋₈-alkanoyl,
C₁₋₈-alkoxy-C₁₋₈-alkoxy,
C₁₋₈-alkoxy-C₁₋₈-alkoxy-C₁₋₈-alkyl,
C₁₋₈-alkoxy-C₁₋₈-alkyl,
(N—C₁₋₈-alkoxy)-C₁₋₈-alkylaminocarbonyl-C₁₋₈-alkoxy,
(N—C₁₋₈-alkoxy)-C₁₋₈-alkylaminocarbonyl-C₁₋₈-alkyl,
C₁₋₈-alkoxy-C₁₋₈-alkylcarbamoyl,
C₁₋₈-alkoxy-C₁₋₈-alkylcarbonyl,
C₁₋₈-alkoxy-C₁₋₈-alkylcarbonylamino,
C₁₋₈-alkoxy-C₁₋₈-alkylheterocyclyl,
C₁₋₈-alkoxycarbonyl,
C₁₋₈-alkoxycarbonyl-C₁₋₈-alkoxy,
C₁₋₈-alkoxycarbonyl-C₁₋₈-alkyl,
C₁₋₈-alkoxycarbonylamino-C₁₋₈-alkoxy,
C₁₋₈-alkoxycarbonylamino-C₁₋₈-alkyl,
C₁₋₈-alkyl,
(N—C₁₋₈-alkyl)-C₁₋₈-alkoxy-C₁₋₈-alkylcarbamoyl,
(N—C₁₋₈-alkyl)-C₁₋₈-alkoxy-C₁₋₈-alkylcarbonylamino,
(N—C₁₋₈-alkyl)-C₁₋₈-alkoxycarbonylamino,
(N—C₁₋₈-alkyl)-C₁₋₈-alkylcarbonylamino-C₁₋₈-alkoxy,
(N—C₁₋₈-alkyl)-C₁₋₈-alkylcarbonylamino-C₁₋₈-alkyl,
(N—C₁₋₈-alkyl)-C₁₋₈-alkylsulfonylamino-C₁₋₈-alkoxy,
(N—C₁₋₈-alkyl)-C₁₋₈-alkylsulfonylamino-C₁₋₈-alkyl,
C₁₋₈-alkylamidinyl,
C₁₋₈-alkylamino-C₁₋₈-alkoxy,
di-C₁₋₈-alkylamino-C₁₋₈-alkoxy,
C₁₋₈-alkylamino-C₁₋₈-alkyl,
di-C₁₋₈-alkylamino-C₁₋₈-alkyl,
C₁₋₈-alkylaminocarbonyl-C₁₋₈-alkoxy,
di-C₁₋₈-alkylaminocarbonyl-C₁₋₈-alkoxy,
C₁₋₈-alkylaminocarbonyl-C₁₋₈-alkoxy-C₁₋₈-alkyl,
C₁₋₈-alkylaminocarbonyl-C₁₋₈-alkyl,
di-C₁₋₈-alkylaminocarbonyl-C₁₋₈-alkyl,
C₁₋₈-alkylaminocarbonylamino-C₁₋₈-alkoxy,
C₁₋₈-alkylaminocarbonylamino-C₁₋₈-alkyl,
C₀₋₈-alkylcarbonylamino,
C₀₋₈-alkylcarbonylamino-C₁₋₈-alkoxy,
C₀₋₈-alkylcarbonylamino-C₁₋₈-alkyl,
C₁₋₈-alkylcarbonyloxy-C₁₋₈-alkoxy,
C₁₋₈-alkylcarbonyloxy-C₁₋₈-alkyl,
C₁₋₈-alkylsulfonyl,
C₁₋₈-alkylsulfonyl-C₁₋₈-alkoxy,
C₁₋₈-alkylsulfonyl-C₁₋₈-alkyl,
C₁₋₈-alkylsulfonylamino-C₁₋₈-alkoxy,
C₁₋₈-alkylsulfonylamino-C₁₋₈-alkyl,
optionally N-mono- or N,N-di-C₁₋₈-alkylated amino,
aryl-C₀₋₈-alkoxy,
aryl-C₀₋₈-alkyl,
optionally N-mono- or N,N-di-C₁₋₈-alkylated carbamoyl-C₀₋₈-alkoxy,
optionally N-mono- or N,N-di-C₁₋₈-alkylated carbamoyl-C₀₋₈alkyl,
carboxy-C₁₋₈-alkoxy,
carboxy-C₁₋₈-alkoxy-C₁₋₈-alkyl,
carboxy-C₁₋₈-alkyl,
cyano,
cyano-C₁₋₈-alkoxy,
cyano-C₁₋₈-alkyl,
C₃₋₁₂-cycloalkyl-C₁₋₈-alkoxy,
C₃₋₁₂-cycloalkyl-C₁₋₈-alkyl,
C₃₋₁₂-cycloalkylcarbonylamino-C₁₋₈-alkoxy,
C₃₋₁₂-cycloalkylcarbonylamino-C₁₋₈-alkyl,
O,N-dimethylhydroxylamino-C₁₋₈-alkyl,
halogen,
halogen substituted C₁₋₈-alkoxy,
halogen substituted C₁₋₈-alkyl,
heterocyclyl-C₀₋₈-alkoxy,
heterocyclyl-C₀₋₈-alkyl,
heterocyclylcarbonyl,
hydroxy-C₁₋₈-alkoxy-C₁₋₈-alkoxy,
hydroxy-C₁₋₈alkoxy-C₁₋₈-alkyl,
hydroxy-C₁₋₈-alkyl,
oxide and oxo;
where, when R¹ is heterocyclyl and contains at least one saturated carbon atom, this heterocyclyl radical may additionally be substituted at a saturated carbon atom by a C₂₋₈-alkylene chain whose two ends are fixed on this saturated carbon atom and thus form a spirocycle, where one CH₂ group of the alkylene chain may be replaced by oxygen;
R² is phenyl or pyridyl, where the nitrogen atom of the pyridyl is located in the ortho- or meta-position relative to the bond from the pyridyl ring to the remainder of the molecule and where the phenyl or pyridyl is substituted by 1-3 radicals, one of which is located in the para-position relative to the bond from the phenyl or pyridyl ring to the remainder of the molecule, independently selected from the group consisting of
C₁₋₈-alkanoyloxy-C₁₋₈-alkyl,
C₂₋₈-alkenyl,
C₂₋₈-alkenyloxy,
C₂₋₈-alkenyloxy-C₁₋₈-alkyl,
C₁₋₈-alkoxy,
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkoxy-C₁₋₈-alkyl,
C₁₋₈-alkoxy-C₁₋₈-alkoxy-C₁₋₈-alkyl,
C₁₋₈-alkoxy-C₁₋₈-alkyl,
C₁₋₈-alkoxy-C₁₋₈-alkylamino-C₁₋₈-alkyl,
C₁₋₈-alkoxy-C₀₋₈-alkyl-C₃₋₈-cycloalkyl-C₀₋₈-alkoxy-C₁₋₈-alkyl,
C₁₋₈-alkoxy-C₁₋₈-alkylsulfanyl,
C₁₋₈-alkoxy-C₁₋₈-alkylsulfanyl-C₁₋₈-alkyl,
C₁₋₈-alkoxycarbonyl,
C₁₋₈-alkoxycarbonyloxy-C₁₋₈-alkyl,
C₁₋₈-alkoxy-C₃₋₈-cycloalkyl-C₁₋₈-alkyl
C₁₋₈-alkyl,
C₁₋₈-alkylsulfanyl,
C₁₋₈-alkylsulfanyl-C₁₋₈-alkoxy,
C₁₋₈-alkylsulfanyl-C₁₋₈-alkoxy-C₁₋₈-alkyl,
C₁₋₈-alkylsulfanyl-C₁₋₈-alkyl,
C₁₋₈-alkylsulfonyl-C₁₋₈-alkoxy-C₁₋₈-alkyl,
C₁₋₈-alkylsulfonyl-C₁₋₈-alkyl,
C₂₋₈-alkynyl,
optionally substituted C₁₋₈-alkoxy,
optionally N-mono- or N,N-di-C₁₋₈-alkylated amino-C₁₋₈-alkoxy,
optionally N-mono- or N,N-di-C₁₋₈-alkylated amino-carbonyl-C₁₋₈-alkyl,
optionally substituted aryl-C₁₋₈-alkoxy-C₁₋₈-alkoxy,
optionally substituted aryl-heterocyclyl-C₀₋₈-alkoxy, optionally substituted heterocyclyl-heterocyclyl-C₀₋₈-alkoxy,
optionally substituted aryl-C₀₋₈-alkoxy-C₁₋₈-alkoxy,
optionally substituted aryl-C₀₋₈-alkoxy-C₁₋₈-alkoxy-C₁₋₈-alkyl,
carboxy-C₁₋₈-alkyl,
cyano,
cyano-C₁₋₈-alkyl,
C₃₋₈-cycloalkyl-C₀₋₈-alkoxy-C₁₋₈-alkoxy,
C₃₋₈-cycloalkyl-C₀₋₈-alkoxy-C₁₋₈-alkoxy-C₁₋₈-alkyl
C₃₋₈-cycloalkyl-C₀₋₈-alkoxy-C₁₋₈-alkyl,
C₃₋₈-cycloalkyl-C₀₋₈-alkylamino-C₁₋₈-alkyl,
halogen-substituted C₁₋₈-alkoxy,
halogen-substituted C₁₋₈-alkyl,
halogen-substituted C₁₋₈-alkoxy-C₁₋₈-alkoxy-C₁₋₈-alkyl,
heterocyclyl-carbonyl-C₁₋₈-alkyl,
heterocyclyl-C₁₋₈-alkyl,
heterocyclyl-sulfanyl-C₁₋₈-alkoxy-C₁₋₈-alkyl and
heterocyclyl-C₀₋₈-alkoxy-C₁₋₈-alkyl;
and may, in addition to the aforementioned substituents, also be substituted by a maximum of 4 halogens;
R³ is
halogen- and/or hydroxy-substituted C₁₋₈-alkoxy,
optionally halogen- and/or hydroxy-substituted C₁₋₈-alkoxy-C₁₋₈-alkoxy,
optionally halogen- and/or hydroxy-substituted C₁₋₈-alkoxy-C₁₋₈-alkyl,
optionally N—C₁₋₈-alkylated C₁₋₈-alkoxy-C₁₋₈-alkylamino-C₁₋₈-alkoxy,
optionally N—C₁₋₈-alkylated C₁₋₈-alkoxy-C₁₋₈-alkylamino-C₁₋₈-alkyl,
C₁₋₈-alkoxy-C₀₋₈-alkylcarbonyl-C₀₋₈-alkoxy,
C₁₋₈-alkoxycarbonylamino-C₁₋₈-alkyl,
optionally halogen- and/or hydroxy-substituted C₁₋₈-alkyl,
optionally N—C₁₋₈-alkylated C₀₋₈-alkylcarbonylamino,
C₀₋₈-alkylcarbonylamino-C₁₋₈-alkoxy,
optionally N—C₁₋₈-alkylated and/or halogen-substituted C₀₋₈-alkylcarbonylamino-C₁₋₈-alkyl,
C₁₋₈-alkylcarbonyloxy,
C₁₋₈-alkylsulfonyl-C₁₋₈-alkoxy,
C₁₋₈-alkylsulfonyl-C₁₋₈-alkyl,
C₂₋₈-alkynyloxy,
optionally N-mono- or N,N-di-C₁₋₈-alkylated amino-C₁₋₈-alkoxy,
optionally N-mono- or N,N-di-C₁₋₈-alkylated amino-C₁₋₈-alkyl,
optionally N-mono- or N,N-di-C₁₋₈-alkylated amino-C₀₋₈-alkylcarbonyl-C₁₋₈-alkoxy,
optionally N-mono- or N,N-di-C₁₋₈-alkylated amino-C₀₋₈-alkylcarbonyl-heterocyclyl-C₀₋₈-alkyl,
optionally N-mono- or N,N-di-C₁₋₈-alkylated amino-C₂₋₈-alkynyloxy,
optionally N-mono- or N,N-di-C₁₋₈-alkylated and optionally hydroxy-substituted amino-C₀₋₈-alkylcarbonyl-C₀₋₈-alkyl,
N-mono- or N,N-di-C₁₋₈-alkylated aminocarbonyl-C₂₋₈-alkynyloxy,
cyano,
cyano-C₁₋₈-alkoxy,
C₃₋₈-cycloalkyl-C₀₋₈-alkoxy,
optionally halogen-substituted C₃₋₈-cycloalkyl-C₀₋₈-alkylcarbonylamino-C₁₋₈-alkyl,
C₃₋₈-cycloalkyl-carbonyloxy-C₀₋₈-alkyl,
heterocyclyl-C₀₋₈-alkoxy,
heterocyclyl-C₀₋₈-alkyl,
optionally N—C₁₋₈-alkylated heterocyclyl-C₀₋₈-alkylamino-C₀₋₈-alkylcarbonyl-C₀₋₈-alkoxy,
optionally N—C₁₋₈-alkylated heterocyclyl-C₀₋₈-alkylamino-C₀₋₈-alkylcarbonyl-C₀₋₈-alkyl,
optionally halogen-substituted heterocyclyl-C₀₋₈-alkylcarbonylamino-C₁₋₈-alkyl,
heterocyclyl-C₂₋₈-alkynyloxy,
heterocyclylcarbonyl-C₀₋₈-alkoxy,
heterocyclylcarbonyl-C₀₋₈-alkyl,
heterocyclylcarbonyl-C₀₋₈-alkylamino-C₁₋₈-alkyl,
heterocyclyl-carbonyloxy-C₀₋₈-alkyl,
optionally N—C₁₋₈-alkylated hydroxy-C₁₋₈-alkylamino-C₁₋₈-alkyl,
hydroxy-C₀₋₈-alkylcarbonyl-C₁₋₈-alkoxy or
optionally N—C₁₋₈-alkylated and/or halogen-substituted C₀₋₈-alkylcarbonylamino-C₁₋₈-alkoxy;
X is -Alk-, —O-Alk-, -Alk-O—, —O-Alk-O—, —S-Alk-, -Alk-S—, -Alk-NR⁴—, —NR⁴-Alk-, —C(O)—NR⁴—, -Alk-C(O)—NR⁴—, -Alk-C(O)—NR⁴-Alk-, —NR⁴—C(O)—, -Alk-NR⁴—C(O)—, —NR⁴—C(O)-Alk-, -Alk-NR⁴—C(O)-Alk-, —O-Alk-C(O)—NR⁴—, —O-Alk-NR⁴—C(O)—, —S(O)₂—NR⁴— or —S(O)₂—NR⁴Alk-, where Alk is C₁₋₈-alkylene which may optionally be substituted by halogen; and where
R⁴ is hydrogen, C₁₋₈-alkyl, C₁₋₈-alkoxy-C₁₋₈-alkyl, acyl, C₃₋₈-cycloalkyl or aryl-C₁₋₈-alkyl;
or a pharmaceutically acceptable salts thereof.

2. A compound according to claim 1, which corresponds to the general formula (IA) 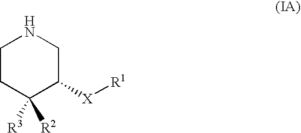
or a pharmaceutically acceptable salt thereof, where the meanings of the substituents R¹, R², R³ and X are as indicated for compounds of the formula (I) according to claim 1.

3. A compound according to claim 1 or 2, wherein
R¹ is
2H-chromenyl,
3,4-dihydro-2H-benzo[1,4]oxazinyl,
3,4-dihydro-2H-benzo[1,4]thiazinyl or
1,3-dihydroindolyl
substituted by 1-3 radicals independently selected from the group consisting of
C₁₋₈-alkoxy,
C₁₋₈-alkoxy-C₁₋₈-alkoxy,
C₁₋₈-alkoxy-C₁₋₈-alkoxy-C₁₋₈-alkyl,
C₁₄-alkoxy-C₁₋₈-alkyl,
C₁₋₈-alkoxy-C₁₋₈-alkylcarbonyl,
C₁₋₈-alkoxycarbonylamino-C₁₋₈-alkoxy,
C₁₋₈-alkoxycarbonylamino-C₁₋₈-alkyl,
C₁₋₈-alkyl,
(N—C₁₋₈-alkyl)-C₀₋₈-alkylcarbonylamino-C₁₋₈-alkoxy,
(N—C₁₋₈-alkyl)-C₀₋₈-alkylcarbonylamino-C₁₋₈-alkyl,
C₀₋₈-alkylcarbonylamino-C₁₋₈-alkoxy,
C₀₋₈-alkylcarbonylamino-C₁₋₈-alkyl,
halogen,
oxo,
halogen-substituted C₁₋₈-alkoxy and
halogen-substituted C₁₋₈-alkyl, or a pharmaceutically acceptable salt thereof.

4. A compound according to any one of claims 1 to 3, wherein
R² is phenyl or pyridyl, where the nitrogen atom of the pyridyl is located in the ortho- or meta-position relative to the bond from the pyridyl ring to the remainder of the molecule and where the phenyl or pyridyl is substituted by one radical located in the para-position relative to the bond from the phenyl or pyridyl ring to the remainder of the molecule, selected from the group consisting of
C₁₋₈-alkoxy,
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkoxy-C₁₋₈-alkyl,
C₁₋₈-alkoxy-C₁₋₈-alkoxy-C₁₋₈-alkyl,
optionally substituted C₁₋₈-alkoxy,
C₁₋₈-alkyl,
C₃₋₈-cycloalkyl-C₀₋₈-alkoxy-C₁₋₈-alkyl,
heterocyclyl-C₀₋₈-alkoxy-C₁₋₈-alkyl,
optionally substituted aryl-heterocyclyl-C₀₋₈-alkoxy and
optionally substituted heterocyclyl-pyrrolidinyl-C₀₋₈-alkoxy or a pharmaceutically acceptable salt thereof.

5. A compound according to claim 1 or 2, wherein
R¹ is 2H-chromenyl or 3,4-dihydro-2H-benzo[1,4]oxazinyl, substituted as defined for a compound of formula (I) according to claim 1;
R² is phenyl, para-substituted, relative to the bond from the phenyl ring to the remainder of the molecule, by one radical selected from the group consisting of
C₁₋₈-alkoxy,
C₁₋₈-alkoxy-C₁₋₈-alkoxy,
C₁₋₈-alkoxy-C₁₋₈-alkoxy-C₁₋₈-alkoxy,
C₁₋₈-alkoxy-C₁₋₈-alkoxy-C₁₋₈-alkyl,
C₁₋₈-alkyl,
C₃₋₈-cycloalkyl-C₀₋₈-alkoxy-C₁₋₈-alkyl,
heterocyclyl-C₀₋₈-alkoxy-C₁₋₈-alkyl,
optionally substituted aryl-heterocyclyl-C₀₋₈-alkoxy and
optionally substituted heterocyclyl-pyrrolidinyl-C₀₋₈-alkoxy;
R³ is
halogen- and/or hydroxy-substituted C₁₋₈-alkoxy,
optionally halogen- and/or hydroxy-substituted C₁₋₈-alkoxy-C₁₋₈-alkoxy,
optionally halogen- or hydroxy-substituted C₁₋₈-alkoxy-C₁₋₈-alkyl,
optionally halogen- and/or hydroxy-substituted C₁₋₈-alkyl,
optionally N—C₁₋₈-alkylated C₁₋₈-alkylcarbonylamino,
optionally N—C₁₋₈-alkylated and/or halogen-substituted C₀₋₈-alkylcarbonylamino-C₁₋₈-alkyl,
C₁₋₈-alkylcarbonyloxy,
optionally N-mono- or N,N-di-C₁₋₈-alkylated amino-C₀₋₈-alkylcarbonyl-C₁₋₈-alkoxy,
cyano,
cyano-C₁₋₈-alkoxy,
C₃₋₈-cycloalkyl-C₀₋₈-alkoxy,
C₃₋₈-cycloalkyl-carbonyloxy-C₀₋₈-alkyl,
heterocyclyl-C₀₋₈-alkoxy or
optionally N—C₁₋₈alkylated and/or halogen-substituted C₀₋₈-alkylcarbonylamino-C₁₋₈-alkoxy;
and
X is -Alk-, —O-Alk- or —O-Alk-O— where Alk is C₁₋₈-alkylene, or a pharmaceutically acceptable salt thereof.

6. A compound of the general formula (I) or (IA) or a pharmaceutically acceptable salt thereof, according to claim 1 or 2, for use as a medicine.

7. (canceled)

8. A method for preventing, for delaying the progression of or for treating high blood pressure, heart failure, glaucoma, myocardial infarction, renal failure, restenoses or stroke, where a therapeutically effective amount of a compound of the general formula (I) or (IA) or a pharmaceutically acceptable salt thereof, according to claim 1 or 2 is used.

9. A pharmaceutical product comprising a compound of the general formula (I) or (IA) or a pharmaceutically acceptable salt thereof, according to claim 1 or 2, and conventional excipients.

10. A pharmaceutical combination in the form of a product or of a kit composed of individual components consisting a) of a compound of the general formula (I) or (IA) or a pharmaceutically acceptable salt thereof, according to claim 1 or 2, and b) at least one pharmaceutical form as active ingredient having a cardiovascular effect.
